(19) United States
(12) Patent Application Publication
(10) Pub. No.: US 2010/0254922 A1
(43) Pub. Date: Oct. 7, 2010

(54) COSMETIC AND/OR DERMATOLOGICAL COMPOSITION BASED ON N,N'-DIARYLMETHYLENEETHYLENEDIAMINEDIACETIC ACID ESTER(S)

(75) Inventors: Cecile KERMORVAN, Saint Maur des Fosses (FR); Veronique CHEVALIER, Villecresnes (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/634,265

(22) Filed: Dec. 9, 2009

Related U.S. Application Data
(60) Provisional application No. 61/193,761, filed on Dec. 22, 2008.
(60) Provisional application No. 61/193,762, filed on Dec. 22, 2008.
(60) Provisional application No. 61/193,763, filed on Dec. 22, 2008.
(60) Provisional application No. 61/193,764, filed on Dec. 22, 2008.

(30) Foreign Application Priority Data
Dec. 15, 2008 (FR) 08 58566
Dec. 15, 2008 (FR) 08 58568
Dec. 15, 2008 (FR) 08 58590
Dec. 15, 2008 (FR) 08 58599

(51) Int. Cl. A61K 8/37; A61Q 17/04; A61Q 19/00

(52) U.S. Cl. 424/60; 514/533

(57) ABSTRACT

The present invention relates to a cosmetic and/or dermatological composition comprising, in a physiologically acceptable medium containing at least one oil:

(a) at least one compound of general formula (Ia):

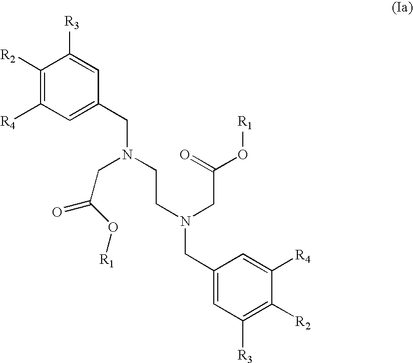

and
(b) an efficient amount of at least one solvent chosen from:
(i) isononyl isononanoate;
(ii) dimethyl isosorbide;
(iii) the amino acid esters of formula (II):

R′₁(CO)N(R′₂)CH(R′₃)(CH₂)ₙ(CO)OR′₄ (II)
(iv) and a mixture thereof.

The present invention relates to cosmetic and/or dermatological compositions comprising at least one N,N′-diarylmethyleneethylenediaminediacetic acid ester and at least one particular solvent.

The compositions according to the invention are in particular intended for caring for and/or making up keratin materials, and especially the skin.

N,N′-Diarylmethyleneethylenediaminediacetic acid esters are described in document WO 94/11338 as iron chelaters and scavengers against the formation of hydroxyl free radicals, making them advantageous for protecting keratin materials, and especially the skin, against oxidative stress caused especially by ultraviolet rays. These derivatives are thus useful in the prevention of cutaneous photoageing.

However, these compounds have the particular feature of being solid at room temperature and of being sparingly soluble or insoluble in the liquid raw materials commonly used in cosmetics and/or dermatology. In particular, these compounds are water-insoluble, and are sparingly soluble in polyols such as glycerol or propylene glycol, and also sparingly soluble in apolar oils, for instance isododecane, hydrogenated polyisobutene (Parleam from NOF Corporation) or cyclopentasiloxane.

Now, it is necessary for these compounds to be formulated in a solubilized form in order to fully exploit their activity, and it is also preferable for their solubilization to be maintained over time in order to avoid any recrystallization during storage of compositions comprising such compounds.

The object of the present invention is, precisely, to propose a novel galenical formulation of N,N′-diarylmethyleneethylenediaminediacetic acid esters that overcomes the abovementioned drawbacks, and thus enables these compounds to be incorporated in a long-lasting dissolved form.

Specifically, the inventors have discovered, unexpectedly, that the combination of certain N,N′-diarylmethyleneethylenediaminediacetic acid esters with at least one particular solvent enables these compounds to be dissolved while avoiding their recrystallization, especially after storage for two months at room temperature (25° C.).

Thus, according to one of its aspects, the invention relates to a cosmetic and/or dermatological composition comprising, in a physiologically acceptable medium containing at least one oil:

(a) at least one compound of general formula (1):

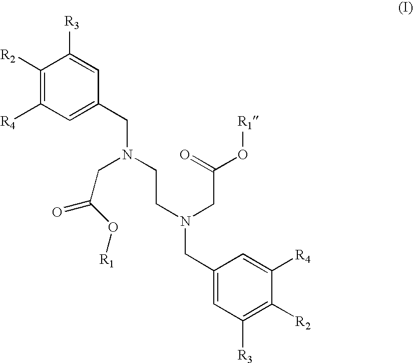

in which:
each group R₁ and R″₁ independently represents a hydrogen atom or a saturated linear alkyl radical containing from 1 to 6 carbon atoms or a saturated branched alkyl radical containing from 3 to 6 carbon atoms, with the proviso that the two groups R₁ and R″₁ do not simultaneously represent a hydrogen atom,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms,
   or salts thereof, and
   (b) an effective amount of at least one solvent chosen from:
(i) isononyl isononanoate;
(ii) dimethyl isosorbide;
(iii) amino acid esters of formula (II):

R′₁(CO)N(R′₂)CH(R′₃)(CH₂)ₙ(CO)OR′₄ (II)

in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical, and
R′₄ represents a linear or branched C₁ to C₁₀ alkyl radical, a linear or branched C₂ to C₁₀ alkenyl radical or a sterol residue; and
(iv) a mixture thereof.

As specified hereinbelow, the said compound of general formula (1) is advantageously present in the compositions in accordance with the invention in a dissolved form.

The compositions in accordance with the invention are advantageously used for caring for and/or making up keratin materials, and especially the skin.

The combination of an N,N′-diarylmethyleneethylenediaminediacetic acid ester in accordance with the invention with such a solvent makes it possible to obtain cosmetic or dermatological compositions that are remarkably effective for the abovementioned applications.

The compositions in accordance with the invention may especially be intended for treating and/or protecting human keratin materials, in particular the skin, against ageing caused especially by exposure to sunlight (ultraviolet rays).

Thus, according to another of its aspects, the present invention relates to a non-therapeutic treatment process for caring for and/or making up keratin materials, especially the skin, comprising at least the step of applying to the said keratin materials, and especially to the skin, at least one composition as defined previously.

### N,N′-diarylmethyleneethylenediaminediacetic Acid Ester

The N,N′-diarylmethyleneethylenediaminediacetic acid esters under consideration according to the invention are compounds of general formula (1):

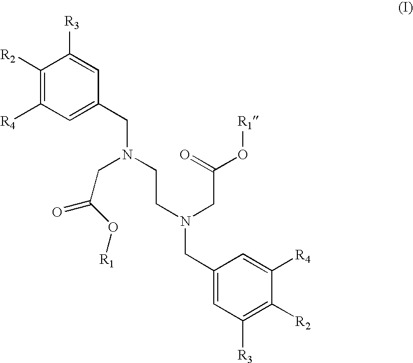

in which:
each group R₁ and R″₁ independently represents a hydrogen atom or a saturated linear alkyl radical containing from 1 to 6 carbon atoms or a saturated branched alkyl radical containing from 3 to 6 carbon atoms, with the proviso that the two groups R₁ and R″₁ do not simultaneously represent a hydrogen atom,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms.

These compounds may be obtained via any method known to those skilled in the art, for example according to the preparation processes described in document WO 94/11338.

According to a first embodiment, they are more particularly N,N′-diarylmethyleneethylenediaminediacetic acid esters of general formula (Ia):

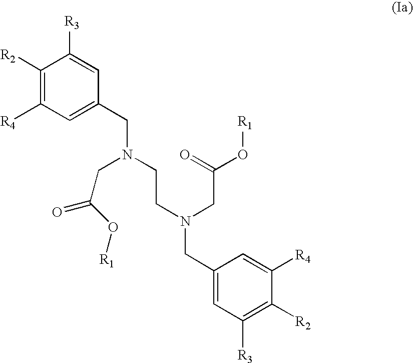

in which:
R₁ represents a saturated linear alkyl radical containing from 1 to 6 carbon atoms or a saturated branched alkyl radical containing from 3 to 6 carbon atoms,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms.

According to one embodiment, R₂, R₃ and R₄ represent a hydrogen atom.

According to another embodiment, R₁ denotes an isopropyl radical.

It may especially be the compound of general formula (Ia) in which R₂, R₃ and R₄ represent a hydrogen atom and R₁ denotes an isopropyl radical, i.e. the diisopropyl ester of N,N′-bis(benzyl)ethylenediamine-N,N′-diacetic acid of formula:

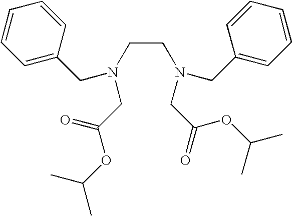

This compound is more particularly described in Example 16 of document WO 94/11338.

According to a second embodiment, they may also be N,N′-diarylmethyleneethylenediaminediacetic acid esters of general formula (Ib):

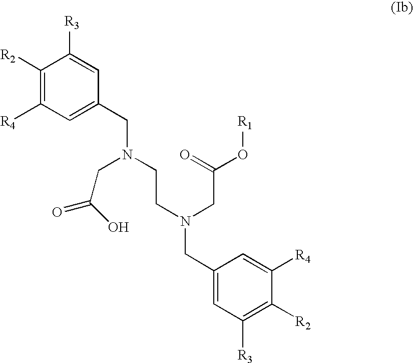

in which:
R₁ represents a saturated linear alkyl radical containing from 1 to 6 carbon atoms or a saturated branched alkyl radical containing from 3 to 6 carbon atoms,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms,

The compounds of formula (Ib) are novel compounds not described in the prior art. The invention also relates to such compounds.

A compound of formula (Ib) that may be mentioned is the isopropyl ester of N,N′-bis(benzyl)ethylenediamine-N,N′-diacetic acid.

The compounds of formula (Ib) may be obtained according to the synthetic scheme I by reacting a dibenzylated ethylenediamine (A) with one molar equivalent of tert-butyl bromoacetate in the presence of a base, for instance potassium carbonate, in a polar solvent, for instance dimethylformamide, and then by reacting one molar equivalent of the bromoacetate derived from the alcohol R₁OH. The intermediate diester (B) obtained is then selectively hydrolysed at its tert-butyl part in acidic medium (for example hydrochloric acid or trifluoroacetic acid) to give the hemiester of formula (Ib).

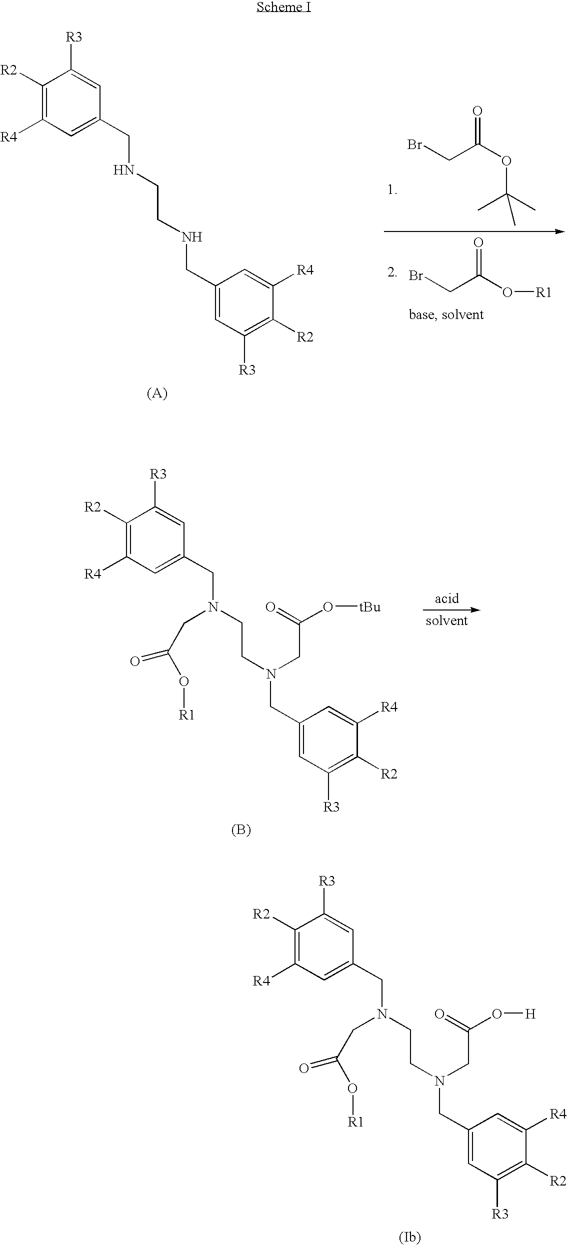

The salts of compounds (I), (Ia) and (Ib) described previously include conventional non-toxic salts of the said compounds, such as those formed from organic or mineral acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphoric acid groups. They may be linear, branched or cyclic aliphatic acids, or alternatively aromatic acids. These acids may also include one or more heteroatoms chosen from O and N, for example in the form of hydroxyl groups. Mention may be made especially of propionic acid, acetic acid, trifluoroacetic acid, terephthalic acid, citric acid and tartaric acid.

The preferred salts are those obtained from hydrochloric acid, sulfuric acid, acetic acid, trifluoroacetic acid, tartaric acid and citric acid.

The compositions in accordance with the invention may comprise from 0.01% to 5% by weight, preferably from 0.1% to 2.5% by weight and especially from 0.5% to 1% by weight of compound of general formula (I), (Ia) or (Ib), in particular of formula (Ia), relative to the total weight of the said composition.

Solvent

The compositions in accordance with the invention comprise a sufficient amount of at least one solvent chosen from:
(i) isononyl isononanoate;
(ii) dimethyl isosorbide;
(iii) amino acid esters of formula (II):

R′₁(CO)N(R′₂)CH(R′₃)(CH₂)ₙ(CO)OR′₄ (II)

in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical, and
R′₄ represents a linear or branched C₁ to C₁₀ alkyl radical, a linear or branched C₂ to C₁₀ alkenyl radical or a sterol residue; and
(iv) a mixture thereof.

According to a first embodiment of the invention, the solvent is isononyl isononanoate.

According to a second embodiment of the invention, the solvent is dimethyl isosorbide.

According to a third embodiment, the solvent is an amino acid ester of formula (II) as indicated previously.

The amino acid esters that are suitable for use in the invention, and the process for synthesizing them, are especially described in patent applications EP 1 044 676 and

EP 0 928 608 from the company Ajinomoto Co.

In the amino acid esters of formula (II), the group R′₁(CO)— is preferably an acyl group of an acid preferably chosen from the group formed by capric acid, lauric acid, myristic acid, palmitic acid, stearic acid, behenic acid, linoleic acid, linolenic acid, oleic acid, isostearic acid, 2-ethylhexanoic acid, coconut oil fatty acids and palm kernel oil fatty acids. These fatty acids may also contain a hydroxyl group. Even more preferably, it will be lauric acid.

The —N(R′₂)CH(R′₃)(CH₂)ₙ(CO)— part of the amino acid ester is preferably chosen from the following amino acids: glycine, alanine, valine, leucine, isoleucine, serine, threonine, proline, hydroxyproline, β-alanine, aminobutyric acid, aminocaproic acid, sarcosine, N-methyl-β-alanine.

Even more preferably, it will be sarcosine.

The part of the amino acid esters corresponding to the group OR′₄ may be obtained from alcohols chosen from the group formed by methanol, ethanol, propanol, isopropanol, butanol, tert-butanol, isobutanol, 3-methyl-1-butanol, 2-methyl-1-butanol, fusel oil, pentanol, hexanol, cyclohexanol, octanol, 2-ethylhexanol, decanol, lauryl alcohol, myristyl alcohol, cetyl alcohol, cetostearyl alcohol, stearyl alcohol, oleyl alcohol, behenyl alcohol, jojoba alcohol, 2-hexadecyl alcohol, 2-octyldodecanol and isostearyl alcohol.

These amino acid esters may be obtained in particular from natural sources of amino acids. In this case, the amino acids originate from the hydrolysis of natural plant proteins (oat, wheat, soybean, palm or coconut) and then necessarily lead to mixtures of amino acids that subsequently need to be esterified and then N-acylated. The preparation of such amino acids is more particularly described in patent application FR 2 796 550, which is incorporated herein by reference.

The amino acid ester more particularly preferred for its use in the present invention is isopropyl N-lauroylsarcosinate of formula CH₃—(CH₂)₁₀CO—N(CH₃)—CH₂—COO—CH(CH₃)₂.

An example of an isopropyl N-lauroylsarcosinate that may be mentioned is the product sold by the company Ajinomoto under the reference Eldew SL-205®.

In the context of the present invention, the expression “effective amount of solvent or of solvent mixture” in accordance with the invention means a sufficient amount of this solvent or solvent mixture to dissolve the N,N′-diarylmethyleneethylene-diaminediacetic acid ester of formula (I), (Ia) or (Ib) and thus to prevent any recrystallization, especially during storage.

For obvious reasons, the amount of solvent in accordance with the invention that is sufficient to dissolve the N,N′-diarylmethyleneethylenediaminediacetic acid ester is liable to vary within a wide range as a function especially of the chemical nature and/or the amount of the said N,N′-diarylmethyleneethylenediaminediacetic acid ester to be dissolved and of the nature of the solvent or solvent mixture used. Adjusting the amount of solvent in accordance with the invention forms part of the competence of a person skilled in the art.

It is clear that, in the embodiments in which this solvent features among one of the three abovementioned solvents or a mixture thereof, the combined presence of another solvent may be envisaged, with the proviso, of course, that this additional compound does not harm the solubility afforded by the required solvent according to the invention.

As an illustration, the solvent according to the invention may be present in an amount ranging from 0.05% to 25% by weight, more preferentially from 0.5% to 12.5% by weight and more particularly from 2.5% to 5% by weight relative to the total weight of the composition.

According to one embodiment, the solvent in accordance with the invention and the compound of general formula (I), (Ia) or (Ib) may be present in a [solvent/compound of general formula (I), (Ia) or (Ib)] mass ratio at least equal to 1.5, especially ranging from 1.5 to 15 and preferably ranging from 1.5 to 10.

According to one embodiment, the solvent in accordance with the invention and the compound of general formula (Ia) may be present in a [solvent/compound of general formula (Ia)] mass ratio at least equal to 1.5, especially ranging from 1.5 to 15 and preferably ranging from 1.5 to 10.

When the solvent is isononyl isononanoate, the [isononyl isononanoate/compound of general formula (I), (Ia) or (Ib)] mass ratio may especially be at least equal to 5.25, for example ranging from 5.25 to 8 and preferably ranging from 5.25 to 6.5.

In particular, the [isononyl isononanoate/compound of general formula (Ia)] mass ratio may especially be at least equal to 5.25, for example ranging from 5.25 to 8 and preferably ranging from 5.25 to 6.5.

When the solvent is dimethyl isosorbide, the [dimethyl isosorbide/compound of general formula (I), (Ia) or (Ib)] mass ratio may especially be at least equal to 1.6, for example ranging from 1.6 to 10 and preferably ranging from 1.6 to 6.

In particular, the [dimethyl isosorbide/compound of general formula (Ia)] mass ratio may especially be at least equal to 1.6, for example ranging from 1.6 to 10 and preferably ranging from 1.6 to 6.

When the solvent is an amino acid ester of formula (II), the [amino acid ester of formula (II)/compound of general formula (I), (Ia) or (Ib)] mass ratio may be especially at least equal to 4.5, for example ranging from 4.5 to 10 and preferably ranging from 4.5 to 6.

In particular, the [amino acid ester of formula (II)/compound of general formula (Ia)] mass ratio may especially be at least equal to 4.5, for example ranging from 4.5 to 10 and preferably ranging from 4.5 to 6.

In particular, the [isopropyl N-lauroylsarcosinate/diisopropyl ester of N,N′-bis(benzyl)ethylene diamine-N,N′-diacetic acid] mass ratio may be at least equal to 4.5, for example ranging from 4.5 to 10 and preferably ranging from 4.5 to 6.

Physiologically Acceptable Medium

The compositions used according to the invention contain a physiologically acceptable medium, i.e. a medium that is compatible with cutaneous tissues such as the skin and the scalp.

Besides the solvents in accordance with the invention mentioned previously, this physiologically acceptable medium comprises at least one oil other than the solvents described previously.

As oils that may be used in the composition of the invention, examples that may be mentioned include:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;
synthetic esters and ethers, especially of fatty acids, for instance the oils of formulae R₁COOR₂ and R₁OR₂ in which R₁ represents a fatty acid residue containing from 8 to 29 carbon atoms and R₂ represents a branched or unbranched hydrocarbon-based chain containing from 3 to 30 carbon atoms, for instance Purcellin oil, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons, of mineral or synthetic origin, such as volatile or non-volatile liquid paraffins, and derivatives thereof, petroleum jelly, polydecenes, and hydrogenated polyisobutene such as Parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
silicone oils, for instance volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, these groups containing from 2 to 24 carbon atoms; phenylsilicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes;
mixtures thereof.

In the list of oils mentioned above, the term “hydrocarbon-based oil” means any oil mainly comprising carbon and hydrogen atoms, and possibly ester, ether, carboxylic acid and/or alcohol groups.

The composition according to the invention may comprise substances that are solid at room temperature (25° C.), for instance fatty acids containing from 8 to 30 carbon atoms, such as stearic acid, lauric acid, palmitic acid and oleic acid; waxes such as lanolin, beeswax, carnauba wax or candelilla wax, paraffin waxes, microcrystalline waxes, ceresin or ozokerite, and synthetic waxes such as polyethylene waxes and Fischer-Tropsch waxes.

These fatty substances may be chosen in a varied manner by a person skilled in the art in order to prepare a composition having the desired properties, for example in terms of consistency or texture.

The composition according to the invention may comprise water and optionally a physiologically acceptable organic solvent chosen, for example, from lower alcohols containing from 1 to 8 carbon atoms and in particular from 1 to 6 carbon atoms, for instance ethanol, isopropanol, propanol or butanol; polyethylene glycols containing from 6 to 80 ethylene oxide units, and polyols, for instance propylene glycol, isoprene glycol, butylene glycol, glycerol and sorbitol.

Advantageously, the compositions according to the invention may be in the form of an emulsion, especially an oil-in-water emulsion, a water-in-oil emulsion, a W/O/W or O/W/O triple emulsion, dispersions of a fatty phase in an aqueous phase with the aid of spherules, these spherules possibly being polymer nanoparticles such as nanospheres or nanocapsules, or lipid vesicles of ionic and/or nonionic type (liposomes, niosomes or oleosornes). These compositions are prepared according to the usual methods.

In addition, the compositions according to the invention may be more or less fluid and may have the appearance of a white or coloured cream, an ointment, a milk, a lotion, a serum, a paste or a mousse. They may be optionally applied to the skin in aerosol form. They may also be in solid form, for example in the form of a stick.

According to one particular embodiment of the invention, the composition according to the invention is a water-in-oil (W/O) or oil-in-water (O/W) emulsion, preferably an oil-in-water emulsion. The proportion of the oily phase of the emulsion may range from 5% to 80% by weight and preferably from 5% to 50% by weight relative to the total weight of the composition.

The emulsions generally contain at least one emulsifier chosen from amphoteric, anionic, cationic and nonionic emulsifiers, used alone or as a mixture, and optionally a co-emulsifier. The emulsifiers are chosen in an appropriate manner according to the emulsion to be obtained (W/O or O/W) emulsion. The emulsifier and the co-emulsifier are generally present in the composition in a proportion ranging from 0.3% to 30% by weight and preferably from 0.5% to 20% by weight relative to the total weight of the composition.

For the W/O emulsions, examples of emulsifiers that may be mentioned include dimethicone copolyols such as the mixture of cyclomethicone and of dimethicone copolyol sold under the name DC 5225 C by the company Dow Corning, and alkyl dimethicone copolyols such as the laurylmethicone copolyol sold under the name Dow Corning 5200 Formulation Aid by the company Dow Corning and the cetyl dimethicone copolyol sold under the name Abil EM 90® by the company Goldschmidt.

Surfactants for W/O emulsions that may also be used include a crosslinked elastomeric solid organopolysiloxane comprising at least one oxyalkylene group, such as those obtained according to the procedure of Examples 3, 4 and 8 of document U.S. Pat. No. 5,412,004 and of the examples of document U.S. Pat. No. 5,811,487, especially the product of Example 3 (synthesis example) of patent U.S. Pat. No. 5,412,004 and such as that sold under the reference KSG 21 by the company Shin-Etsu.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; and mixtures thereof such as the mixture of glyceryl stearate and of PEG-40 stearate.

In a known manner, the cosmetic or dermatological composition for the use according to the invention may also contain adjuvants that are common in cosmetics or dermatology, such as gelling agents, film-forming polymers, preserving agents, fragrances, fillers, UV-screening agents, bactericides, odour absorbers, dyestuffs, plant extracts, cosmetic and dermatological active agents, and salts. The amounts of these various adjuvants are those conventionally used in the field under consideration, for example from 0.01% to 20% of the total weight of the composition. Depending on their nature, these adjuvants may be introduced into the fatty phase and/or into the aqueous phase.

The composition according to the invention may also comprise at least one organic photoprotective agent and/or at least one mineral photoprotective agent that is active in the UVA and/or UVB range (absorbers), which may be water-soluble or liposoluble, or else insoluble in the commonly used cosmetic solvents.

Preferably, a system that screens out both UVA radiation and UVB radiation will be used.

Sunscreens are molecules that absorb UV radiation and thus prevent this radiation from reaching skin cells. They may absorb either mainly UVB or mainly UVA, depending on their nature. There are two major categories of sunscreen, either organic, or mineral (zinc oxide or titanium oxide). By using them in cosmetic compositions in combination and in sufficient amount, they block a large proportion of the UV radiation.

However, it is commonly accepted that in order to be efficient, these formulations must be used under good application conditions (sufficient amount, frequent renewal, uniform spreading). Users do not always comply with these application conditions, which increases the risk of an appreciable amount of UV radiation reaching the skin cells and thus of giving rise to the biological effects mentioned above. Furthermore, in order to obtain absorption with respect to the entire wavelength range of the UVB+UVA solar UV spectrum, several molecules that absorb in complementary wavelength ranges need to be combined.

The additional organic screening agents are chosen especially from anthranilates; cinnamic derivatives; salicylic derivatives; camphor derivatives; benzophenone derivatives; [β,β-d]phenylacrylate derivatives; triazine derivatives; benzotriazole derivatives; benzalmalonate derivatives, especially those mentioned in patent U.S. Pat. No. 5,624,663; benzimidazole derivatives; imidazolines; bis-benzazolyl derivatives as described in patents EP 0 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives as described in patent applications EP 0 832 642, EP 1 027 883, EP 1 300 137 and DE 101 62 844; screening polymers and screening silicones such as those described especially in patent application WO 93/04665; α-alkylstyrene-based dimers, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981; merocyanin derivatives such as those described in patent applications WO 04/006 878, WO 05/058 269 and WO 06/032 741; and mixtures thereof.

As examples of additional organic photoprotective agents, mention may be made of those denoted hereinbelow under their INCI name:

Cinnamic Derivatives:

Ethylhexyl methoxycinnamate sold in particular under the trade name Parsol MCX by DSM Nutritional Products, Inc.,

Isopropyl methoxycinnamate,

Isoamyl methoxycinnamate sold under the trade name Neo Heliopan E 1000 by Symrise,

DEA methoxycinnamate,

Diisopropyl methylcinnamate,

Glyceryl ethylhexanoate dimethoxycinnamate.

para-Aminobenzoic Acid Derivatives:

PABA,

Ethyl PABA,

Ethyl dihydroxypropyl PABA,

Ethylhexyl dimethyl PABA sold in particular under the name Escalol 507 by ISP,

Glyceryl PABA,

PEG-25 PABA sold under the name Uvinul P25 by BASF.

Salicylic Derivatives:

Homosalate sold under the name Eusolex HMS by Rona/EM Industries,

Ethylhexyl salicylate sold under the name Neo Heliopan OS by Symrise,

Dipropylene glycol salicylate sold under the name Dipsal by Scher,

TEA salicylate sold under the name Neo Heliopan TS by Symrise.

β,β-Diphenylacrylate Derivatives:

Octocrylene sold in particular under the trade name Uvinul N539 by BASF,

Etocrylene sold in particular under the trade name Uvinul N35 by BASF.

Benzophenone Derivatives:

Benzophenone-1 sold under the trade name Uvinul 400 by BASF,

Benzophenone-2 sold under the trade name Uvinul D50 by BASF,

Benzophenone-3 or Oxybenzone sold under the trade name Uvinul M40 by BASF,

Benzophenone-4 sold under the trade name Uvinul MS40 by BASF,

Benzophenone-5,

Benzophenone-6 sold under the trade name Helisorb 11 by Norquay,

Benzophenone-8 sold under the trade name Spectra-Sorb UV-24 by American Cyanamid,

Benzophenone-9 sold under the trade name Uvinul DS-49 by BASF,

Benzophenone-12

n-hexyl 2-(4-diethylamino-2-hydroxybenzoyl)benzoate sold under the trade name Uvinul A+by BASF.

Benzylidenecamphor Derivatives:

3-Benzylidenecamphor manufactured under the name Mexoryl SD by Chimex,

4-Methylbenzylidenecamphor sold under the name Eusolex 6300 by Merck,

Benzylidenecamphorsulfonic acid manufactured under the name Mexoryl SL by Chimex,

Camphor benzalkonium methosulfate manufactured under the name Mexoryl SO by Chimex,

Terephthalylidenedicamphorsulfonic acid manufactured under the name Mexoryl SX by Chimex,

Polyacrylamidomethylbenzylidenecamphor manufactured under the name Mexoryl SW by Chimex.

Phenylbenzimidazole Derivatives:

Phenylbenzimidazolesulfonic acid sold in particular under the trade name Eusolex 232 by Merck,

Disodium phenyl dibenzimidazole tetrasulfonate sold under the trade name Neo Heliopan AP by Symrise.

Phenylbenzotriazole Derivatives:

Drometrizole trisiloxane sold under the name Silatrizole by Rhodia Chimie,

Methylenebis(benzotriazolyl)tetramethylbutylphenol sold in solid form under the trade name MIXXIM BB/100 by Fairmount Chemical, or in micronized form as an aqueous dispersion under the trade name Tinosorb M by Ciba Specialty Chemicals.

Triazine Derivatives:

Bis(ethylhexyloxyphenol)methoxyphenyltriazine sold under the trade name Tinosorb S by Ciba Geigy,

Ethylhexyltriazone sold in particular under the trade name Uvinul T150 by BASF,

Diethylhexylbutamidotriazone sold under the trade name Uvasorb HEB by Sigma 3V,

2,4,6-tris(dineopentyl 4′-aminobenzalmalonate)-s-triazine

2,4,6-tris(diisobutyl 4′-aminobenzalmalonate)-s-triazine,

2,4-bis(n-butyl 4′-aminobenzoate)-6-(aminopropyltrisiloxane)-s-triazine,

2,4-bis(dineopentyl 4′-aminobenzalmalonate)-6-(n-butyl 4′-aminobenzoate)-s-triazine,

the symmetrical triazine screening agents described in patent U.S. Pat. No. 6,225,467, patent application WO 2004/085 412 (see compounds 6 and 9) or the document Symmetrical Triazine Derivatives IRCOM Journal, IP.COM INC West Henrietta, N.Y., US (20 Sep. 2004), especially 2,4,6-tris(biphenyl)-1,3,5-triazines (in particular 2,4,6-tris(biphenyl-4-yl-1,3,5-triazine) and 2,4,6-tris(terphenyl)-1,3,5-triazine which is also mentioned in Beiersdorf patent applications WO 06/035 000, WO 06/034 982, WO 06/034 991, WO 06/035 007, WO 2006/034 992 and WO 2006/034 985,

Anthranilic Derivatives:

Menthyl anthranilate sold under the trade name Neo Heliopan MA by Haarmann & Reimer.

Imidazoline Derivatives:

Ethylhexyldimethoxybenzylidenedioxoimidazoline propionate.

Benzalmalonate Derivatives:

Polyorganosiloxane containing benzalmalonate functions, for instance Polysilicone-15, sold under the trade name Parsol SLX by DSM Nutritional products, Inc.

4,4-Diarylbutadiene Derivatives:

1,1-Dicarboxy(2,2′-dimethylpropyl)-4,4-diphenylbutadiene

Benzoxazole Derivatives:

2,4-bis[5-(1-dimethylpropyl)benzoxazol-2-yl(4-phenyl)imino]-6-(2-ethylhexyl)imino-1,3,5-triazine sold under the name Uvasorb K2A by Sigma 3V.

Merocyanin Derivatives:

Octyl 5-N,N-diethylamino-2-phenylsulfonyl-2,4-pentadienoate. and mixtures thereof.

The preferred organic photoprotective agents are chosen from:

Ethylhexyl methoxycinnamate,

Ethylhexyl salicylate,

Homosalate,

Octocrylene,

Phenylbenzimidazolesulfonic acid,

Benzophenone-3,

Benzophenone-4,

Benzophenone-5,

n-Hexyl 2-(4-diethylamino-2-hydroxybenzoyl)benzoate,

4-Methylbenzylidenecamphor,

Terephthalylidenedicamphorsulfonic acid,

Disodium phenyldibenzimidazoletetrasulfonate,

Methylenebis(benzotriazolyl)tetramethylbutylphenol,

Bis(ethylhexyloxyphenol)methoxyphenyltriazine,

Ethylhexyltriazone,

Diethylhexylbutamidotriazone,

2,4,6-Tris(dineopentyl 4′-aminobenzalmalonate)-s-triazine,

2,4,6-Tris(diisobutyl 4′-aminobenzalmalonate)-s-triazine,

2,4-Bis(n-butyl 4′-aminobenzoate)-6-(aminopropyltrisiloxane)-s-triazine,

2,4-Bis(dineopentyl 4′-aminobenzalmalonate)-6-(n-butyl 4′-aminobenzoate)-s-triazine,

2,4,6-Tris(biphenyl-4-yl)-1,3,5-triazine,

2,4,6-Tris(terphenyl)-1,3,5-triazine,

Drometrizole trisiloxane,

Polysilicone-15,

1,1-Dicarboxy(2,2′-dimethylpropyl)-4,4-diphenylbutadiene,

2,4-Bis[5-1(dimethylpropyl)benzoxazol-2-yl(4-phenyl)imino]-6-(2-ethylhexyl)imino-1,3,5-triazine,

Octyl 5-N,N-diethylamino-2-phenylsulfonyl-2,4-pentadienoate,

and mixtures thereof.

The organic screening agents in accordance with the invention may represent from 0.1% to 30% and preferably from 1% to 25% of the total weight of the composition.

The additional mineral UV-screening agents used in accordance with the present invention are metal oxide pigments. More preferentially, the mineral UV-screening agents of the invention are metal oxide pigments with a mean elemental particle size of less than or equal to 500 nm, more preferentially between 5 nm and 500 nm and even more preferentially between 10 nm and 100 nm, and preferentially between 15 and 50 nm.

They may be chosen especially from titanium oxide, zinc oxide, iron oxide, zirconium oxide and cerium oxide, or mixtures thereof, and more particularly titanium oxides.

Such coated or uncoated metal oxide pigments are described in particular in patent application EP-A-0 518 773. Commercial pigments that may be mentioned include the products sold by the companies Kemira, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or aluminium) of polyethylene, silicones, proteins (collagen, elastin), alkanolamines, silicon oxides, metal oxides or sodium hexametaphosphate.

The coated pigments are more particularly titanium oxides that have been coated:
with silica, such as the product Sunveil from the company Ikeda,
with silica and iron oxide, such as the product Sunveil F from the company Ikeda,
with silica and alumina, such as the products Microtitanium Dioxide MT 500 SA and Microtitanium Dioxide MT 100 SA from the company Tayca and Tioveil from the company Tioxide,
with alumina, such as the products Tipaque TTO-55 (B) and Tipaque TTO-55 (A) from the company Ishihara and UVT 14/4 from the company Kemira,
with alumina and aluminium stearate, such as the product Microtitanium Dioxide MT 100 T, MT 100 TX, MT 100 Z and MT-01 from the company Tayca, the products Solaveil CT-10 W and Solaveil CT 100 from the company Uniqema and the product Eusolex T-AVO from the company Merck,
with silica, alumina and alginic acid, such as the product MT-100 AQ from the company Tayca,
with alumina and aluminium laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminium stearate and treated with a silicone, such as the product STT-30-DS from the company Titan Kogyo,
with silica and treated with a silicone, such as the product UV-Titan X 195 from the company Kemira,
with alumina and treated with a silicone, such as the products Tipaque TTO-55 (S) from the company Ishihara or UV Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca,
TiO₂ treated with octyltrimethylsilane, sold under the trade name T 805 by the company Degussa Silices,
TiO₂ treated with a polydimethylsiloxane, sold under the trade name 70250 Cardre UF TiO2SI3 by the company Cardre,
anatase/rutile TiO₂ treated with a polydimethylhydrogenosiloxane, sold under the trade name Microtitanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Miyoshi Kasei under the name UFTR, by the company Tomen under the name ITS and by the company Tioxide under the name Tioveil AQ.

The uncoated zinc oxide pigments are, for example those sold under the name Z-Cote by the company Sunsmart.

The coated zinc oxide pigments are, for example:
those sold under the name Zinc Oxide CS-5 by the company Toshibi (ZnO coated with polymethylhydrogenosiloxane);
those sold under the name Daitopersion ZN-30 and Daitopersion ZN-50 by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name NFD Ultrafine ZnO by the company Daikin (ZnO coated with perfluoroalkyl phosphate and copolymer based on perfluoroalkylethyl as a dispersion in cyclopentasiloxane);
those sold under the name SPD-Z1 by the company Shin-Etsu (ZnO coated with silicone-grafted acrylic polymer, dispersed in cyclodimethylsiloxane);
those sold under the name Escalol Z100 by the company ISP (alumina-treated ZnO dispersed in an ethylhexyl methoxycinnamate/PVP-hexadecene/methicone copolymer mixture);
those sold under the name Fuji ZnO-SMS-10 by the company Fuji Pigment (ZnO coated with silica and polymethylsilsesquioxane).

The uncoated cerium oxide pigments are sold under the name Colloidal Cerium Oxide by the company Rhone-Poulenc.

The uncoated iron oxide nanopigments are sold, for example, by the company Mitsubishi under the name TY-220,

The coated iron oxide pigments are sold, for example, by the company BASF under the name Transparent Iron Oxide.

Mention may also be made of mixtures of metal oxides, especially of titanium dioxide and of cerium dioxide, including the silica-coated equal-weight mixture of titanium dioxide and of cerium dioxide, sold by the company Ikeda under the name Sunveil A, and also the alumina, silica and silicone-coated mixture of titanium dioxide and of zinc dioxide, such as the product M 261 sold by the company Kemira, or the alumina, silica and glycerol-coated mixture of titanium dioxide and of zinc dioxide, such as the product M 211 sold by the company Kemira.

According to the invention, coated or uncoated titanium oxide pigments are particularly preferred.

The mineral screening agents in accordance with the invention may represent from 0.5% to 40% and preferably from 1% to 30% of the total weight of the composition.

According to a second of its aspects, the present invention relates to cosmetic and/or dermatological compositions in the form of an oil-in-water emulsion comprising at least one N,N′-diarylmethyleneethylenediaminediacetic acid ester and at least one particular surfactant.

The compositions according to this second aspect of the invention are in particular intended for caring for and/or making up keratin materials, and especially the skin.

As indicated previously, the N,N′-diarylmethyleneethylenediaminediacetic acid esters have the particular feature of being solid at room temperature and of being insoluble or sparingly soluble in the liquid raw materials commonly used in cosmetics and/or dermatology.

This is why, in order to fully exploit their activity, it is generally necessary to formulate these compounds with a particular solvent devoted specifically to dissolving them.

Moreover, formulations in the form of oil-in-water emulsions are advantageous in cosmetics and dermatology in the sense that they generally show very good tolerance on the skin, and have sensory and/or organoleptic properties that are satisfactory to the consumer (especially in terms of feel).

Now, the inventors have found that the preparation of oil-in-water emulsions with anionic surfactants, for instance fatty acids such as stearic acid, give rise to chemical degradation of the ester compounds, leading to a reduction in the amount of these ester compounds present in the emulsion and thus to a loss of efficacy of the cosmetic product.

For these reasons, there is still need for compositions that are suitable for formulating N,N′-diarylmethyleneethylenediaminediacetic acid esters in the form of more efficient oil-in-water emulsions, which do not harm the chemical stability of the said ester, and which achieve all this while at the same time maintaining good skin tolerance.

According to its second aspect, the object of the present invention is, precisely, to propose a novel galenical formulation of N,N′-diarylmethyleneethylenediaminediacetic acid esters that can overcome the abovementioned drawbacks and that can maintain the chemical stability of these compounds durably over time.

Specifically, the inventors have discovered, unexpectedly, that the combination of certain N,N′-diarylmethyleneethylenediaminediacetic acid esters with certain particular surfactants can be satisfactory in these terms, and especially can maintain the chemical stability of the said esters formulated in the form of an oil-in-water emulsion.

In the context of this second aspect of the invention, the invention thus relates, according to one of its aspects, to a cosmetic and/or dermatological composition in the form of an oil-in-water emulsion comprising:

(a) at least one compound of general formula (1):

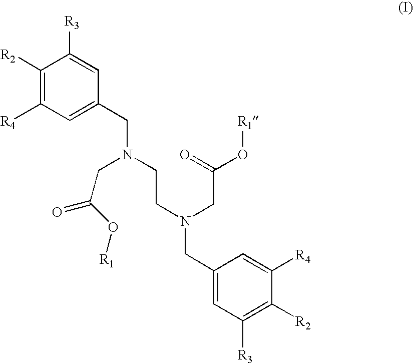

in which:
each group R₁ and R″₁ independently represents a hydrogen atom or a saturated linear alkyl radical containing from 1 to 6 carbon atoms or a saturated branched alkyl radical containing from 3 to 6 carbon atoms, with the proviso that the two groups R₁ and R″₁ do not simultaneously represent a hydrogen atom,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms,
   or salts thereof,
   (b) an effective amount of at least one solvent for the said compound of general formula (I), and
   (c) at least one ionic surfactant chosen from alkali metal salts of (C10-C22)acylglutamic acids and alkali metal salts of palmitoyl sarcosinate, and mixtures thereof.

The compound of general formula (I) is advantageously present, in the compositions in accordance with this second aspect of the invention, in a dissolved form by virtue of the presence of at least one of its solvents in an effective amount. Its chemical stability is maintained by the presence of at least one particular ionic surfactant as defined previously.

The combination, in an oil-in-water emulsion, of a N,N′-diarylmethyleneethylenediaminediacetic acid ester in accordance with the invention with such an ionic surfactant makes it possible to obtain cosmetic and/or dermatological compositions that are remarkably effective for the applications mentioned previously.

The compositions in accordance with this second aspect of the invention may especially be intended for treating and/or protecting human keratin materials, in particular the skin, against the ageing caused especially by exposure to sunlight (ultraviolet rays).

Thus, according to another of its aspects, the present invention relates to a non-therapeutic treatment process for caring for and/or making up keratin materials, especially the skin, comprising at least the step of applying to the said keratin materials, and especially to the skin, at least one composition as defined previously.

The N,N′-diarylmethyleneethylenediaminediacetic acid esters under consideration according to this second aspect of the invention are compounds of general formula (I), (Ia) or (Ib) as defined previously for the first aspect of the invention.

The amount of N,N′-diarylmethyleneethylenediaminediacetic acid esters to be used in a composition according to this second aspect of the invention depends on the desired cosmetic effect and may thus vary within a wide range.

A person skilled in the art can readily, on the basis of his general knowledge, determine the appropriate amounts.

The compositions in accordance with this second aspect of the invention may comprise from 0.2% to 10% by weight, preferably from 0.5% to 5% by weight and especially from 0.8% to 3% by weight, for example at least 1% by weight, of compound of general formula (I), (Ia) or (Ib), in particular of formula (Ia), relative to the total weight of the said composition.

Solvent for the N,N′-diarylmethyleneethyleneDiaminediacetic Acid Ester

The compositions in accordance with this second aspect of the invention comprise at least one N,N′-diarylmethyleneethylenediaminediacetic acid ester of formula (I), (Ia) or (Ib) in combination with at least an effective amount of a solvent therefor.

Suitable solvents that may especially be mentioned include ethanol, hydrogenated isoparaffin, squalane, isopropyl palmitate, 2-octyldodecanol, 2-ethylhexyl salicylate, dipropylene glycol, isopropyl myristate, hexylene glycol, phenyl trimethicone, apricot oil, isononyl isononanoate, dimethyl isosorbide, and the amino acid esters of formula (II):

R′₁(CO)N(R′₂)CH(R′₃)(CH₂)ₙ(CO)OR′₄ (II)

in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical, and
R′₄ represents a linear or branched C₁ to C₁₀ alkyl radical, a linear or branched C₂ to C₁₀ alkenyl radical or a sterol residue; and
   a mixture thereof, It is preferably a solvent chosen from isononyl isononanoate, dimethyl isosorbide and the amino acid esters of formula (II) as defined above, and a mixture thereof.

The solvent under consideration according to this second aspect of the invention may especially be a solvent as defined previously for the first aspect of the invention.

Ionic Surfactant

The compositions in accordance with this second aspect of the invention also comprise at least one ionic surfactant chosen from alkali metal salts of (C10-C22)acylglutamic acids and alkali metal salts of palmitoyl sarcosinate, and mixtures thereof.

Examples of alkali metal salts that may be mentioned include the sodium salts, the potassium salts and the lithium salts, and preferably the sodium salts.

According to one embodiment, the compositions in accordance with this second aspect of the invention comprise at least one alkali metal salt of (C 10-C22)acylglutamic acids and preferably an alkali metal salt of (C12-C20)acylglutamic acids, for example an alkali metal salt of (C16-C18)acylglutamic acids.

Such a salt may especially be an alkali metal salt of stearoylglutamic acid, of lauroylglutamic acid, of a C16 acylglutamic acid, of myristoylglutamic acid, of cocoylglutamic acid or of hydrogenated tallow acylglutamic acid.

Preferably, such a surfactant will be an ionic surfactant chosen from sodium stearoylglutamate, disodium stearoylglutamate, potassium stearoylglutamate, sodium lauroylglutamate, disodium lauroylglutamate, potassium lauroylglutamate, sodium cocoyl-glutamate and hydrogenated tallow sodium acylglutamate, and mixtures thereof, and preferably sodium stearoyl glutamate.

By way of illustration, an example that may be mentioned is the sodium stearoylglutamate sold by the company Ajinomoto under the reference Amisoft HS11 PF®.

According to another embodiment, the compositions in accordance with this second aspect of the invention comprise at least one alkali metal salt of palmitoyl sarcosinate.

It will preferably be sodium palmitoyl sarcosinate,

For obvious reasons, the amount of ionic surfactant in accordance with this second aspect of the invention is liable to vary within a wide range as a function especially of the chemical nature and/or the amount of the said N,N′-diarylmethyleneethylenediaminediacetic acid ester and of the nature of the ionic surfactant used. Adjusting the amount of ionic surfactant in accordance with this second aspect of the invention forms part of the competence of a person skilled in the art.

By way of illustration, the ionic surfactant in accordance with this second aspect of the invention may be present in an amount of active material ranging from 0.01% to 20% by weight, more preferentially from 0.1% to 10% by weight, even more preferentially from 0.2% to 5% by weight and even more particularly from 0.25% to 2% by weight relative to the total weight of the composition.

Emulsion

The compositions in accordance with this second aspect of the invention are in the form of an oil-in-water emulsion.

Besides an ionic surfactant in accordance with this second aspect of the invention, these emulsions may contain at least one additional emulsifier chosen from amphoteric, anionic, cationic and nonionic emulsifiers, used alone or as a mixture. The additional emulsifiers are obviously chosen in a manner appropriate for obtaining an oil-in-water emulsion.

As additional emulsifiers that may be used, examples that may be mentioned include nonionic emulsifiers such as fatty acid esters of oxyalkylenated (more particularly polyoxyethylenated) polyols, for example polyethylene glycol stearates, for instance PEG-100 stearate, PEG-50 stearate and PEG-40 stearate; and mixtures thereof such as the mixture of glyceryl monostearate and polyethylene glycol stearate (100 EO) sold under the name Simulsol 165 by the company SEPPIC; fatty acid esters of oxyalkylenated sorbitan comprising, for example, from 20 to 100 EO, for example those sold under the trade names Tween 20 and Tween 60 by the company Uniqema; oxyalkylenated (oxyethylenated and/or oxypropylenated) fatty alcohol ethers; oxyalkylenated (oxyethylenated and/or oxypropylenated) fatty alcohol ethers; sugar esters, for instance sucrose stearate; and mixtures thereof, for instance the mixture of glyceryl stearate and PEG-100 stearate, sold under the name Arlacel 165 by the company Uniqema, or the mixture of glyceryl stearate and PEG-40 stearate.

Emulsions free of additional emulsifying surfactants or containing less than 0.5% thereof relative to the total weight of the composition may also be prepared, by using suitable compounds, for example polymers with emulsifying properties such as acrylic acid/stearyl methacrylate polymers, such as those sold under the names Carbopol 1342 and Pemulen by the company Noveon; or 2-acrylamido-2-methylpropanesulfonic acid polymers and copolymers, optionally crosslinked and/or neutralized, for instance the poly(2-acrylamido-2-methylpropanesulfonic acid) sold by the company Clariant under the name Hostacerin AMPS (CTFA name: ammonium polyacryldimethyltauramide) or the acrylamide/sodium 2-acrylamidomethylpropanesulfonate polymer, such as the product sold under the name Sepigel 305 by the company SEPPIC (INCI name: polyacrylamide/C13-C14 isoparaffin/laureth-7), the (AMPS/ethoxylated C12/C14 alcohol methacrylate) copolymer (8 mol EO) (80/20) (Aristoflex LNC® from Clariant); ionic or nonionic polymer particles, more particularly anionic polymer particles especially such as isophthalic acid or sulfoisophthalic acid polymers, and in particular phthalate/sulfoisophthalate/glycol copolymers (for example diethylene glycol/phthalate/isophthalate/1,4-cyclohexanedimethanol (INCI name: diglycol/CHDM/isophthalates/SIP copolymer) sold under the name Eastman AQ Polymer (AQ35S, AQ38S, AQ55S or AQ48 Ultra) by the company Eastman Chemical.

The composition according to this second aspect of the invention may also comprise at least one organic photoprotective agent and/or at least one mineral photoprotective agent that is active in the UVA and/or UVB range (absorbers), which may be water-soluble or liposoluble, or else insoluble in the commonly used cosmetic solvents, as defined previously for the first aspect of the invention.

The second aspect of the invention is more particularly illustrated by Examples 7 and 8.

According to a third of its aspects, the present invention relates to cosmetic and/or dermatological compositions comprising at least one N,N′-diarylmethyleneethylenediaminediacetic acid ester and at least one particular emulsifing polymer.

The compositions according to this third aspect of the invention are in particular intended for caring for and/or making up keratin materials, and especially the skin.

As indicated previously, the N,N′-diarylmethyleneethylenediaminediacetic acid esters have the particular feature of being solid at room temperature and of being insoluble or sparingly soluble in the liquid raw materials commonly used in cosmetics and/or dermatology.

Now, it is necessary for these compounds to be formulated in a dissolved or dispersed form in order to fully exploit their activity, and it is also preferable for this state to be maintained over time so as to avoid any recrystallization during storage of the compositions comprising such compounds.

According to a third of its aspects, the present invention is directed towards formulating N,N′-diarylmethyleneethylenediaminediacetic acid esters in the oily phase of an oil-in-water or water-in-oil-in-water emulsion.

Specifically, the inventors have discovered, unexpectedly, that the combination of certain N,N′-diarylmethyleneethylenediaminediacetic acid esters with at least one particular emulsifing polymer allows these ester compounds to be introduced into the oily phase of this type of emulsion while avoiding their recrystallization, especially after storage for two months at room temperature (25° C.).

In the context of this third aspect of the invention, the invention thus relates, according to one of its aspects, to a cosmetic and/or dermatological composition in the form of an oil-in-water emulsion or a water-in-oil-in-water emulsion, containing in its oily phase:

(a) at least one compound of general formula (1):

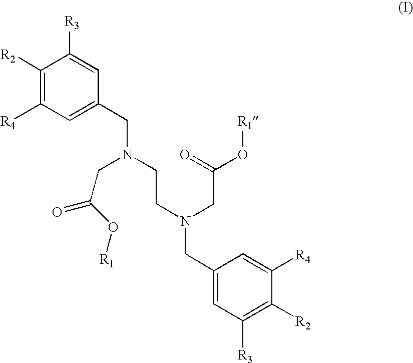

in which:
each group R₁ and R″₁ independently represents a hydrogen atom or a saturated linear alkyl radical containing from 1 to 6 carbon atoms or a saturated branched alkyl radical containing from 3 to 6 carbon atoms, with the proviso that the two groups R₃ and R″₁ do not simultaneously represent a hydrogen atom,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms,
   or salts thereof, and

(b) an effective amount of at least one amphiphilic polymer comprising at least one acrylamido-2-methylpropanesulfonic acid (AMPS) unit.

As emerges from Examples 9 to 12 below, the presence of an amphiphilic polymer in accordance with this third aspect of the invention effectively ensures the presence of the compound of general formula (1) in the oily phase of the emulsion.

The use for this purpose of such an amphiphilic polymer thus advantageously makes it possible to dispense with the use of solubilizing solvents, thereby simplifying the formulation of emulsions containing the ester compounds.

The compositions in accordance with this third aspect of the invention are advantageously used for caring for and/or making up keratin materials, and especially the skin.

The combination of an N,N′-diarylmethyleneethylenediaminediacetic acid ester in accordance with the invention with such an amphiphilic polymer makes it possible to obtain cosmetic and/or dermatological compositions that are remarkably effective for the applications mentioned previously.

The compositions in accordance with this third aspect of the invention may especially be intended for treating and/or protecting human keratin materials, in particular the skin, against the ageing caused especially by exposure to sunlight (ultraviolet rays).

Thus, according to another of its aspects, the present invention relates to a non-therapeutic treatment process for caring for and/or making up keratin materials, especially the skin, comprising at least the step of applying to the said keratin materials, and especially to the skin, at least one composition as defined previously.

The N,N′-diarylmethyleneethylenediaminediacetic acid esters under consideration according to this third aspect of the invention are compounds of general formula (I), (Ia) or (Ib) as defined previously for the first aspect of the invention.

The amount of N,N′-diarylmethyleneethylenediaminediacetic acid esters to be used in a composition according to this third aspect of the invention depends on the desired cosmetic effect and may thus vary within a wide range.

A person skilled in the art can, on the basis of his general knowledge, readily determine the appropriate amounts.

The compositions in accordance with this third aspect of the invention may especially comprise from 0.2% to 10% by weight, preferably from 0.5% to 5% by weight and especially from 0.8% to 3% by weight, for example at least 1% by weight, of compound of general formula (1), (Ia) or (Ib), in particular of formula (Ia), relative to the total weight of the said composition.

Emulsifying Polymers

For the purposes of the invention, the terms “emulsifying polymer” and “amphiphilic polymer” are intended to denote a polymer having amphiphilic properties, i.e. having at least one hydrophilic part and at least one hydrophobic part. Hydrophilic groups and hydrophobic groups are well known to those skilled in the art.

For the purposes of the present invention, the term “polymer” is intended to denote a compound comprising at least two repeating units and in particular at least five repeating units.

The amphiphilic polymers under consideration according to this third aspect of the invention are amphiphilic polymers comprising at least one acrylamido-2-methylpropanesulfonic acid (AMPS) unit.

The amphiphilic polymers comprising at least one acrylamido-2-methyl-propanesulfonic acid (AMPS) unit that may be used in this third aspect of the present invention, which are also known more simply as “amphiphilic AMPS polymers” hereinbelow, comprise both a hydrophilic part and a hydrophobic part comprising at least one fatty chain.

The amphiphilic AMPS polymers according to this third aspect of the invention are especially chosen from amphiphilic polymers of at least one 2-acrylamidomethylpropanesulfonic acid (AMPS) monomer and of at least one ethylenically unsaturated comonomer comprising at least one hydrophobic part containing from 7 to 30 carbon atoms, in particular from 7 to 22 carbon atoms or even from 12 to 22 carbon atoms.

The amphiphilic AMPS polymers according to this third aspect of the invention generally have a weight-average molecular weight ranging from 50 000 to 10 000 000 g/mol, in particular from 100 000 to 8 000 000 g/mol and even more particularly from 100 000 to 7 000 000 g/mol.

They may be crosslinked or non-crosslinked.

When the amphiphilic AMPS polymers according to this third aspect of the invention are crosslinked, the crosslinking agents may be chosen from the polyolefinically unsaturated compounds commonly used for the crosslinking of polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebis(acrylamide), methylenebis(methacrylamide), triallylamine, triallyl cyanurate, diallyl maleate, tetraallylethylenediamine, tetraallyloxyethane, trimethyloipropane diallyl ether, allyl (meth)acrylate, allylic ethers of alcohols of the sugar series, or other allylic or vinyl ethers of polyfunctional alcohols, and also allylic esters of phosphoric and/or vinylphosphonic acid, or mixtures of these compounds.

The crosslinking agents may be chosen especially from methylenebis(acrylamide), allyl methacrylate and trimethylolpropane triacrylate (TMPTA).

The degree of crosslinking may range, for example, from 0.01 mol % to 10 mol % and preferably from 0.2 mol % to 2 mol % relative to the polymer.

The amphiphilic AMPS polymers according to this third aspect of the invention may be chosen especially from statistical amphiphilic AMPS polymers modified by reaction with a C₆-C₂₂ n-monoalkylamine or di-n-alkylamine such as those described in patent application WO 00/31154.

An amphiphilic polymer that is suitable for use in this third aspect of the invention may comprise at least one ethylenically unsaturated hydrophilic monomer chosen, for example, from acrylic acid, methacrylic acid or substituted alkyl derivatives thereof or esters thereof obtained with monoalkylene or polyalkylene glycols, acrylamide, methacrylamide, vinylpyrrolidone, vinylformamide, maleic anhydride, itaconic acid or maleic acid, or mixtures thereof.

An amphiphilic polymer according to this third aspect of the invention may comprise at least one ethylenically unsaturated hydrophobic comonomer.

An amphiphilic polymer that is suitable for use in this third aspect of the invention may comprise at least one hydrophobic part chosen from saturated or unsaturated, linear alkyl radicals, for instance n-octyl, n-decyl, n-hexadecyl, n-dodecyl and oleyl, branched alkyl radicals, for instance isostearyl, or cyclic alkyl radicals, for instance cyclododecane or adamantane.

An amphiphilic AMPS polymer may also contain at least one ethylenically unsaturated hydrophobic comonomer comprising, for example:
a fluoro or C₇-C₁₈ fluoroalkyl radical (for example the group of formula —(CH₂)₂—(CF₂)₉—CF₃),
a cholesteryl radical or a cholesterol-based radical (for example cholesteryl hexanoate),
a polycyclic aromatic group, for instance naphthalene or pyrene,
a silicone, alkylsilicone or alkylfluorosilicone radical.

These copolymers are especially described in document EP-A-0 750 899, patent U.S. Pat. No. 5,089,578 and in the following publications by Yotaro Morishima:
“Self-assembling amphiphilic polyelectrolytes and their nanostructures—Chinese Journal of Polymer Science Vol. 18, No. 40, (2000), 323-336”;
“Micelle formation of random copolymers of sodium 2-(acrylamido)-2-methylpropanesulfonate and a nonionic surfactant macromonomer in water as studied by fluorescence and dynamic light scattering —Macromolecules 2000, Vol. 33, No. 10-3694-3704”;
“Solution properties of micelle networks formed by nonionic moieties covalently bound to a polyelectrolyte: salt effects on rheological behavior—Langmuir, 2000, Vol. 16, No. 12, 5324-5332”;
“Stimuli responsive amphiphilic copolymers of sodium 2-(acrylamido)-2-methylpropanesulfonate and associative macromonomers—Polym. Preprint, Div. Polym. Chem. 1999, 40(2), 220-221”.

They are also described in documents EP 1 069 142, WO 02/44224, WO 02/44225, WO 02/44227, WO 02/44229, WO 02/44230, WO 02/44231, WO 02/44267, WO 02/44268, WO 02/44269, WO 02/44270, WO 02/44271, WO 02/43677, WO 02/43686, WO 02/43687, WO 02/43688 and WO 02/43689, in the name of Clariant.

An ethylenically unsaturated hydrophobic comonomer of the invention may preferably be chosen from the acrylates or acrylamides of formula (1) below:

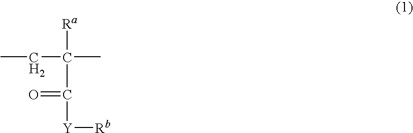

in which:
R^{a} denotes a hydrogen atom or a linear or branched C₁-C₆ alkyl radical, preferably methyl;
Y denotes O or NH;
R^{b} denotes a hydrophobic radical comprising a fatty chain containing from 7 to 30 carbon atoms, preferably from 7 to 22 and more particularly from 12 to 22 carbon atoms.

The hydrophobic radical R^{b} is chosen from saturated or unsaturated linear C₇-C₂₂ alkyl radicals (for example n-octyl, n-decyl, n-hexadecyl, n-dodecyl or oleyl), branched alkyl radicals (for example isostearie) or cyclic alkyl radicals (for example cyclo-dodecane or adamantane); C₇-C₁₅ alkylperfluoro radicals (for example the group of formula —(CH₂)₂—(CF₂)₉—CF₃); the cholesteryl radical or a cholesterol ester, for instance cholesteryl hexanoate; aromatic polycyclic groups, for instance naphthalene or pyrene.

Among these radicals, linear and branched alkyl radicals are more particularly preferred.

According to one preferred form of the invention, the hydrophobic radical R^{b} may also comprise at least one alkylene oxide unit and preferably a polyoxyalkylene chain.

The polyoxyalkylene chain may preferentially consist of ethylene oxide units and/or propylene oxide units and even more particularly consists solely of ethylene oxide units.

The number of mol of oxyalkylene units may generally range from 1 to 30 mol, more preferably from 1 to 25 mol and even more preferably from 3 to 20 mol.

Among these polymers that may be mentioned are
crosslinked or non-crosslinked, neutralized or non-neutralized copolymers comprising from 15% to 60% by weight of AMPS units and from 40% to 85% by weight of (C₈-C₁₆)alkyl)-meth)acrylamide units or of (C₈-C₁₆)alkyl (meth)acrylate units relative to the polymer, such as those described in patent application EP-A-0 750 899;
terpolymers comprising from 10 mol % to 90% of acrylamide units, from 0.1 mol % to 10 mol % of AMPS units and from 5 mol % to 80 mol % of n-(C₆-C₁₈)alkylacrylamide units relative to the polymer, such as those described in U.S. Pat. No. 5,089,578;
partially or totally neutralized non-crosslinked copolymers of AMPS and of n-dodecyl, n-hexadecyl or n-octadecyl methacrylate, such as those described in the Morishima articles mentioned above;
non-crosslinked and crosslinked copolymers of partially or totally neutralized AMPS and of n-dodecylmethacrylamide, such as those described in the Morishima articles mentioned above.

Amphiphilic AMPS polymers that may also be mentioned include copolymers of totally neutralized AMPS and of n-dodecyl, n-hexadecyl and/or n-octadecyl methacrylate, and also non-crosslinked and crosslinked copolymers of AMPS and of n-dodecylmethacrylamide.

Mention will be made more particularly of crosslinked or non-crosslinked amphiphilic AMPS copolymers consisting of:

(a) 2-acrylamido-2-methylpropanesulfonic acid (AMPS) units of formula (2) below:

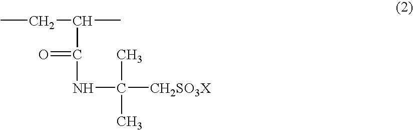

in which X is a proton, an alkali metal cation, an alkaline-earth metal cation or an ammonium ion;

(b) and units of formula (3) below:

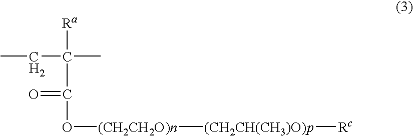

in which n and p, independently of one another, denote a number of mol and range from 0 to 30, preferably from 1 to 25 and more preferably from 3 to 20, with the proviso that n+p is less than or equal to 30, preferably less than 25 and better still less than 20; R^{a} denotes a hydrogen atom or a linear or branched C₁-C₆ alkyl radical, preferably methyl, and R^{c} denotes a linear or branched alkyl containing from 7 to 22 and preferably from 12 to 22 carbon atoms.

In formula (2), the cation X more particularly denotes sodium or ammonium.

Among the monomers of formula (3) that may be mentioned are:
esters of (meth)acrylic acid and of a C₁₀-C₁₈ fatty alcohol polyoxyethylenated with 8 EO, for instance the product Genapol C-080® sold by the company Clariant,
esters of (meth)acrylic acid and of a C₁₁ fatty oxo alcohol polyoxyethylenated with 8 EO, for instance the product Genapol UD-080® sold by the company Clariant,
esters of (meth)acrylic acid and of a C₁₂-C₁₄ polyoxyethylenated fatty alcohol with 7 EO for instance the product Genapol LA-070® sold by the company Clariant,
esters of (meth)acrylic acid and of a C₁₂-C₁₄ polyoxyethylenated fatty alcohol with 11 EO for instance the product Genapol LA-110® sold by the company Clariant,
esters of (meth)acrylic acid and of a C₁₆-C₁₈ polyoxyethylenated fatty alcohol with 8 EO for instance the product Genapol T-080® sold by the company Clariant,
esters of (meth)acrylic acid and of a C₁₆-C₁₈ polyoxyethylenated fatty alcohol with 15 EO for instance the product Genapol T-150® sold by the company Clariant,
esters of (meth)acrylic acid and of a C₁₆-C₁₈ polyoxyethylenated fatty alcohol with 11 EO for instance the product Genapol T-110® sold by the company Clariant,
esters of (meth)acrylic acid and of a C₁₆-C₁₈ polyoxyethylenated fatty alcohol with 20 EO, for instance the product Genapol T-200® sold by the company Clariant,
esters of (meth)acrylic acid and of a C₁₆-C₁₈ polyoxyethylenated fatty alcohol with 25 EO for instance the product Genapol T-250® sold by the company Clariant,
esters of (meth)acrylic acid and of a C₁₈-C₂₂ polyoxyethylenated fatty alcohol with 25 EO and/or of a C₁₆-C₁₈ polyoxyethylenated fatty isoalcohol with 25 EO.

The products that will be chosen more particularly are:

(i) non-crosslinked products for which p=0, n=7 or 25, R^{a} denotes methyl and R^{e} represents a C₁₂-C₁₄ or C₁₆-C₁₈ alkyl mixture,

(ii) crosslinked products for which p=0, n=8 or 25, R^{a} denotes methyl and R^{c} represents a C₁₆-C₁₈ alkyl mixture.

These polymers are described and synthesized in patent application EP 1 069 142.

These particular amphiphilic AMPS polymers may be obtained according to the standard processes of free-radical polymerization in the presence of one or more initiators, for instance azobisisobutyronitrile (AIBN), azobisdimethylvaleronitrile, 2,2-azobis(2-amidinopropane) hydrochloride (ABAH), organic peroxides such as dilauryl peroxide, benzoyl peroxide, tert-butyl hydroperoxide, etc., mineral peroxide compounds such as potassium or ammonium persulfate, or H₂O₂ optionally in the presence of reducing agents.

These amphiphilic AMPS polymers may be obtained especially by free-radical polymerization in tert-butanol medium, in which they precipitate. By using precipitation polymerization in tert-butanol, it is possible to obtain a size distribution of the polymer particles that is particularly favourable for its uses.

The reaction may be performed at a temperature of between 0 and 150° C. and preferably between 10 and 100° C., either at atmospheric pressure or under reduced pressure.

It may also be performed under inert atmosphere and preferably under nitrogen.

The amphiphilic AMPS polymers according to this third aspect of the invention may preferably be partially or totally neutralized with a mineral base such as sodium hydroxide, potassium hydroxide, aqueous ammonia or an organic base such as monoethanolamine, diethanolamine, triethanolamine, an aminomethylpropanediol, N-methylglucamine, basic amino acids, for instance arginine and lysine, and mixtures of these compounds. They may especially be totally or almost totally neutralized, i.e. at least 80% neutralized.

The molar percentage concentration of the units of formula (2) and of the units of formula (3) in the amphiphilic AMPS polymers according to this third aspect of the invention may vary as a function of the desired cosmetic application, the nature of the emulsion (oil-in-water or water-in-oil emulsion) and the rheological properties of the desired formulation. It can for example range between 0.1 and 99.9 mol %.

The distribution of the monomers in the amphiphilic AMPS polymers according to this third aspect of the invention may be, for example, alternate, block (including multiblock) or random.

As a guide, and without this being limiting, mention may be made especially of the copolymer of AMPS and of ethoxylated C₁₂-C₁₄ alcohol methacrylate (non-crosslinked copolymer obtained from Genapol LA-070 and from AMPS) (CTFA name: Ammonium Acryloyldimethyltaurate/Laureth-7 methacrylate copolymer) sold under the name Aristoflex LNC by the company Clariant, the copolymer of AMPS and of ethoxylated (25 BO) stearyl methacrylate (copolymer crosslinked with trimethylolpropane triacrylate, obtained from Genapol T-250 and from AMPS) (CTFA name: Ammonium Acryloyldimethyltaurate/Steareth-25 Methacrylate Crosspolymer) sold under the name Aristoflex HMS by the company Clariant, Aristoflex SNC (80/20 copolymer of AMPS/ethoxylated (8 mol EO) C₁₆-C₁₈ alcohol methacrylate; CTFA name: Ammonium Acryloyldimethyltaurate/Steareth-8 methacrylate copolymer) and Aristoflex HMB (copolymer of AMPS/ethoxylated (25 EO) behenyl methacrylate, crosslinked with trimethylolpropane triacrylate (TMPTA)).

In the context of this third aspect of the present invention, the term” effective amount of amphiphilic AMPS polymer” in accordance with the invention means a sufficient amount of this polymer to form the N,N′-diarylmethylene-ethylenediaminediacetic acid ester of formula (I), (Ia) or (Ib) in the oily phase of the emulsion in a long-lasting form and thus prevent any recrystallization, especially during storage.

For obvious reasons, the amount of amphiphilic AMPS polymer in accordance with this third aspect of the invention, which is sufficient to formulate the N,N′-diarylmethyleneethylenediaminediacetic acid ester in the oily phase of the emulsion is liable to vary within a wide range as a function especially of the chemical nature and/or of the amount of the said N,N′-diarylmethyleneethylenediaminediacetic acid ester to be formulated and of the structure of the polymer used. Adjusting the amount of amphiphilic AMPS polymer in accordance with this third aspect of the invention forms part of the competence of a person skilled in the art.

As an illustration, the amphiphilic AMPS polymer according to this third aspect of the invention may be present in an amount of active material ranging from 0.05% to 10% by weight, more preferentially from 0.1% to 5% by weight and even more particularly from 0.5% to 3% by weight relative to the total weight of the composition.

According to one embodiment, the amphiphilic AMPS polymer in accordance with this third aspect of the invention and the compound of general formula (I), (Ia) or (Ib) may be present in an [amphiphilic AMPS polymer/compound of general formula (I), (Ia) or (Ib)] mass ratio ranging from 0.1 to 3 and especially from 0.5 to 2, for example from 0.5 to 1.5, or even equal to 1.

According to one embodiment, the amphiphilic AMPS polymer in accordance with this third aspect of the invention and the compound of general formula (Ia) may be present in an [amphiphilic AMPS polymer/compound of general formula (Ia)] mass ratio ranging from 0.1 to 3 and especially from 0.5 to 2, for example from 0.5 to 1.5, or even equal to 1.

Physiologically Acceptable Medium

The compositions used according to this third aspect of the invention contain a physiologically acceptable medium, i.e. a medium that is compatible with cutaneous tissues such as the skin and the scalp.

This physiologically acceptable medium comprises at least one oily phase and at least one aqueous phase.

As oils that may be used in the oily phase of the composition of this third aspect of the invention, examples that may be mentioned include:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;
synthetic esters and ethers, especially of fatty acids, for instance the oils of formulae R₁COOR₂ and R₁OR₂ in which R₁ represents a fatty acid residue containing from 8 to 29 carbon atoms and R₂ represents a branched or unbranched hydrocarbon-based chain containing from 3 to 30 carbon atoms, for instance Purcellin oil, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons, of mineral or synthetic origin, such as volatile or non-volatile liquid paraffins, and derivatives thereof, petroleum jelly, polydecenes, and hydrogenated polyisobutene such as Parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
silicone oils, for instance volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, these groups containing from 2 to 24 carbon atoms; phenylsilicones, for instance phenyl trimethicones, phenyl dimethicones, phenyltrimethylsiloxydiphenylsiloxanes, diphenyl dimethicones, diphenylmethyl-diphenyltrisiloxanes or 2-phenylethyl trimethylsiloxy silicates, and polymethylphenyl-siloxanes;
mixtures thereof.

In the list of oils mentioned above, the term “hydrocarbon-based oil” means any oil mainly comprising carbon and hydrogen atoms, and possibly ester, ether, carboxylic acid and/or alcohol groups.

The oily phase of the composition according to this third aspect of the invention may also comprise substances that are solid at room temperature (25° C.), for instance fatty acids containing from 8 to 30 carbon atoms, such as stearic acid, lauric acid, palmitic acid and oleic acid; waxes such as lanolin, beeswax, carnauba wax or candelilla wax, paraffin waxes, microcrystalline waxes, ceresin or ozokerite, and synthetic waxes such as polyethylene waxes and Fischer-Tropsch waxes.

These fatty substances may be chosen in a varied manner by a person skilled in the art in order to prepare a composition having the desired properties, for example in terms of consistency or texture.

The composition according to this third aspect of the invention comprises at least one aqueous phase, which may comprise water and optionally a physiologically acceptable organic solvent chosen, for example, from lower alcohols containing from 1 to 8 carbon atoms and in particular from 1 to 6 carbon atoms, for instance ethanol, isopropanol, propanol or butanol; polyethylene glycols containing from 6 to 80 ethylene oxide units, and polyols, for instance propylene glycol, isoprene glycol, butylene glycol, glycerol and sorbitol.

As indicated previously, the compositions according to this third aspect of the invention are in the form of an oil-in-water (O/W) emulsion or a water-in-oil-in-water (W/O/W) triple emulsion. These compositions are prepared according to the usual methods.

In addition, the compositions according to this third aspect of the invention may be more or less fluid and may have the appearance of a white or coloured cream, an ointment, a milk, a lotion, a serum, a paste or a mousse. They may be optionally applied to the skin in aerosol form. They may also be in solid form, for example in the form of a stick.

According to one particular embodiment of the invention, the composition according to this third aspect of the invention is an oil-in-water (O/W) emulsion.

The proportion of the oily phase of the emulsion may range from 5% to 80% by weight and preferably from 5% to 50% by weight relative to the total weight of the emulsion.

Besides the amphiphilic AMPS polymer in accordance with this third aspect of the invention, the emulsions may contain at least one other emulsifier (additional emulsifier) chosen from amphoteric, anionic, cationic and nonionic emulsifiers, used alone or as a mixture. The emulsifiers are chosen in an appropriate manner according to the emulsion to be obtained (W/O/W or O/W). The additional emulsifier is generally present in the composition in a proportion ranging from 0.3% to 30% by weight and preferably from 0.5% to 20% by weight relative to the total weight of the composition.

For the W/O/W emulsions, examples of emulsifiers that may be mentioned include dimethicone copolyols such as the mixture of cyclomethicone and dimethicone copolyol, sold under the name DC 5225 C by the company Dow Corning, and alkyl dimethicone copolyols such as lauryl methicone copolyol sold under the name Dow Corning 5200 Formulation Aid by the company Dow Corning and cetyl dimethicone copolyol sold under the name Abil EM 90® by the company Goldschmidt. A crosslinked elastomeric solid organopolysiloxane comprising at least one oxyalkylene group, such as those obtained according to the procedure of Examples 3, 4 and 8 of document U.S. Pat. No. 5,412,004 and the examples of document U.S. Pat. No. 5,811,487, especially the product of Example 3 (synthetic example) of U.S. Pat. No. 5,412,004, such as the product sold under the reference KSG 21 by the company Shit-Etsu, may also be used as surfactants for W/O/W emulsions.

For the O/W emulsions, examples of emulsifiers that may be mentioned include nonionic emulsifiers such as fatty acid esters of oxyalkylenated (more particularly polyoxyethylenated) glycerol; fatty acid esters of oxyalkylenated sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-40 stearate.

In a known manner, the cosmetic and/or dermatological composition of this third aspect of the invention may also contain adjuvants that are common in cosmetics and/or dermatology, such as gelling agents, film-forming polymers, preserving agents, fragrances, fillers, UV-screening agents, bactericides, odour absorbers, dyestuffs, plant extracts, and cosmetic and dermatological active agents, and salts thereof. The amounts of these various adjuvants are those conventionally used in the field under consideration, for example from 0.01% to 20% of the total weight of the composition. Depending on their nature, these adjuvants may be introduced into the fatty phase and/or into the aqueous phase.

The composition according to this third aspect of the invention may also comprise at least one organic photoprotective agent and/or at least one mineral photoprotective agent that is active in the UVA and/or UVB range (absorbers), which may be water-soluble or liposoluble, or else insoluble in the commonly used cosmetic solvents, as defined previously for the first aspect of the invention.

This third aspect of the invention is more particularly illustrated by Examples 9 to 12.

According to a fourth of its aspects, the present invention relates to cosmetic and/or dermatological compositions in the form of an oil-in-water emulsion that is in the form of oleosomes and that comprises at least one N,N′-diarylmethylene-ethylenediaminediacetic acid ester and at least one particular surfactant system.

The compositions according to this fourth aspect of the invention are in particular intended for caring for and/or making up keratin materials, and especially the skin.

As indicated previously, the N,N′-diarylmethyleneethylenediaminediacetic acid esters have the particular feature of being solid at room temperature and of being insoluble or sparingly soluble in the liquid raw materials commonly used in cosmetics and/or dermatology.

This is why, in order to fully exploit their activity, it is generally necessary to formulate these compounds with a particular solvent devoted specifically towards dissolving them.

Moreover, formulations in the form of oil-in-water emulsions are advantageous in cosmetics and dermatology since they generally have very good tolerance on the skin, and sensory and/or organoleptic properties that are satisfactory to the consumer (especially in terms of feel).

Now, the inventors have found that the production of oil-in-water emulsions with anionic surfactants, for instance fatty acids such as stearic acid, give rise to chemical degradation of the ester compounds, inducing a reduction in the amount of these ester compounds present in the emulsion and thus a loss of efficacy of the cosmetic product.

For these reasons, there is still a need for compositions that are suitable for formulating N,N′-diarylmethyleneethylenediaminediacetic acid esters in the form of more efficient oil-in-water emulsions, which do not require the additional presence of particular solvents for the said ester and which do not harm the chemical stability of the said ester.

According to a fourth of its aspects, the object of the present invention is, precisely, to propose a novel galenical formulation of N,N′-diarylmethyleneethylene-diaminediacetic acid esters that can overcome the abovementioned drawbacks and that can especially maintain the chemical stability of these compounds durably over time.

Specifically, the inventors have discovered, unexpectedly, that the combination of an oil-in-water emulsion of certain N,N′-diarylmethyleneethylenediaminediacetic acid esters with a particular surfactant system can be satisfactory in these terms.

Such a formulation can especially maintain the chemical stability of the said ester present in the oil-in-water emulsion.

It also makes it possible to dispense with the use of particular solvents for these ester compounds.

In the context of this fourth aspect of the invention, the invention thus relates, according to one of its aspects, to a cosmetic and/or dermatological composition in the form of an oil-in-water emulsion formed from oily globules with a mean diameter of less than 0.8 micrometres and dispersed in an aqueous phase, characterized in that each oily globule:
comprises at least one compound of general formula (1):

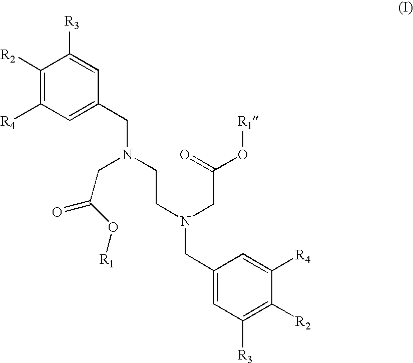

in which:
   each group R₁ and R″₁ independently represents a hydrogen atom or a saturated linear alkyl radical containing from 1 to 6 carbon atoms or a saturated branched alkyl radical containing from 3 to 6 carbon atoms, with the proviso that the two groups R₁ and R″₁ do not simultaneously represent a hydrogen atom,
   R₂, R₃ and R₄ represent, independently of each other, a hydrogen atom or a radical —OR₅, and
   R₅ represents a hydrogen atom or a saturated linear alkyl radical containing from 1 to 5 carbon atoms or a saturated branched alkyl radical containing from 3 to 5 carbon atoms,
   or salts thereof, and
is individually coated with a monolamellar or multilamellar liquid crystal layer obtained from at least one lipophilic nonionic surfactant, from at least one hydrophilic nonionic surfactant and from at least one ionic surfactant chosen from alkali metal salts of (C10-C22)acylglutamic acids, alkali metal salts of cetyl phosphate and alkali metal salts of palmitoyl sarcosinate, and mixtures thereof.

The chemical stability of compound (I) is preserved especially by virtue of the presence of at least one particular ionic surfactant as defined previously.

The combination, in an oil-in-water emulsion in the form of oleosomes, of an N,N′-diarylmethyleneethylenediaminediacetic acid ester in accordance with the invention with such a surfactant system makes it possible to obtain cosmetic and/or dermatological compositions that are remarkably effective for the applications mentioned previously. The ester thus formulated does not recrystallize over time and the emulsion thus remains uniform.

The compositions in accordance with this fourth aspect of the invention may be intended especially for treating and/or protecting human keratin materials, in particular the skin, against ageing caused especially by exposure to sunlight (ultraviolet rays).

Thus, according to another of its aspects, the present invention relates to a non-therapeutic treatment process for caring for and/or making up keratin materials, especially the skin, comprising at least the step of applying to the said keratin materials, and especially to the skin, at least one composition as defined previously.

The N,N′-diarylmethyleneethylenediaminediacetic acid esters under consideration according to this fourth aspect of the invention are compounds of general formula (I), (Ia) or (Ib) as defined previously for the first aspect of the invention.

The amount of N,N′-diarylmethyleneethylenediaminediacetic acid esters to be used in a composition according to this fourth aspect of the invention depends on the desired cosmetic effect and may thus vary within a wide range.

A person skilled in the art can, on the basis of his general knowledge, readily determine the appropriate amounts.

The compositions in accordance with this fourth aspect of the invention may comprise from 0.2% to 10% by weight, preferably from 0.5% to 5% by weight and especially from 0.8% to 3% by weight, for example at least 1% by weight, of compound of general formula (I), (Ia) or (Ib), in particular of formula (Ia), relative to the total weight of the said composition.

Oil-in-water Emulsion in the Form of Oleosomes

The compositions in accordance with this fourth aspect of the invention are in the form of oleosomes,

Oleosomes consist of an oil-in-water emulsion formed by oily globules provided with a lamellar liquid crystal coating dispersed in an aqueous phase.

In the context of this fourth aspect of the present invention, it is more particularly an emulsion of the oil-in-water type formed from oily globules each provided with a lamellar liquid crystal coating and dispersed in an aqueous phase, characterized in that each oily globule is individually coated with a monolamellar or multilamellar layer obtained from at least one lipophilic nonionic surfactant, from at least one hydrophilic nonionic surfactant and from at least one ionic surfactant as defined previously, the coated oily globules having a mean diameter of less than 0.8 micrometre.

The mean size of the coated oily globules is less than 0.8 micrometre and preferably 0.6 micrometre, and especially greater than 0.150 micrometre.

The mean size of the coated oily globules may be expressed as a numerical mean size measured especially using a granulometer of Brookhaven BI90Plus® type, the measuring principle of which is based on quasi-elastic light scattering (QELS).

As indicated previously, the surfactant system comprises at least one ionic surfactant chosen from alkali metal salts of (C10-C22)acylglutamic acids, alkali metal salts of cetyl phosphate and alkali metal salts of palmitoyl sarcosinate, and mixtures thereof.

Examples of alkali metal salts that may be mentioned include the sodium salts, the potassium salts and the lithium salts, and preferably the sodium salts.

According to one embodiment, the compositions in accordance with this fourth aspect of the invention comprise at least one alkali metal salt of (C10-C22)acylglutamic acids and preferably an alkali metal salt of (C12-C20)acylglutamic acids, for example an alkali metal salt of (C16-C18)acylglutamic acids.

Such a salt may especially be one of the alkali metal salts of stearoylglutamic acid, of lauroylglutamic acid, of a C₁₋₆ acylglutamic acid, of myristoylglutamic acid, of cocoylglutamic acid or of hydrogenated tallow acylglutamic acid.

Such a surfactant will preferably be an ionic surfactant chosen from sodium stearoylglutamate, disodium stearoylglutamate, potassium stearoylglutamate, sodium lauroylglutamate, disodium lauroylglutamate, potassium lauroylglutamate, sodium cocoylglutamate and hydrogenated tallow sodium acylglutamate, and mixtures thereof, and preferably sodium stearoylglutamate.

As an illustration, an example that may be mentioned is the sodium stearoylglutamate sold by the company Ajinomoto under the reference Amisoft HS11 PF®.

According to another embodiment, the compositions in accordance with this fourth aspect of the invention comprise at least one alkali metal salt of cetyl phosphate.

It will preferably be potassium cetyl phosphate.

As an illustration, an example that may be mentioned is the potassium cetyl phosphate sold by the company DSM Nutritional Products under the reference Amphisol K®.

According to another embodiment, the compositions in accordance with this fourth aspect of the invention comprise at least one alkali metal salt of palmitoyl sarcosinate.

It will preferably be sodium palmitoyl sarcosinate.

For obvious reasons, the amount of ionic surfactant in accordance with this fourth aspect of the invention is liable to vary within a wide range as a function especially of the nature of the ionic surfactant used. Adjusting the amount of ionic surfactant in accordance with this fourth aspect of the invention forms part of the competence of a person skilled in the art.

As an illustration, the ionic surfactant in accordance with this fourth aspect of the invention may be present in an amount of active material ranging from 0.1% to 20% by weight, more preferentially from 0.15% to 10% by weight, even more preferentially from 0.2% to 5% by weight and even more particularly from 0.25% to 2% by weight relative to the total weight of the composition.

According to one preferential embodiment of this fourth aspect of the invention, the lipophilic nonionic surfactant and the hydrophilic nonionic surfactant each comprise at least one saturated fatty chain containing from 12 to 22 carbon atoms approximately. Even more preferentially, this fatty chain contains from 16 to 22 carbon atoms.

According to another preferential embodiment of this fourth aspect of the invention, the lipophilic nonionic surfactant has an HLB of between about 2 and about 5. As is well known, the term “HLB (Hydrophilic-Lipophilic Balance)” means the equilibrium between the size and force of the hydrophilic group and the size and force of the lipophilic group of the surfactant.

Examples of such nonionic lipophilic surfactants are sucrose distearate, diglyceryl distearate, sucrose tristearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glycerol ester of palmitic and stearic acids, the monostearate polyoxyethylenated with 2 EO (comprising 2 oxyethylene units), glyceryl mono- and dibehenate, and pentaerythrityl tetrastearate.

According to another preferential embodiment of this fourth aspect of the invention, the hydrophilic nonionic surfactant preferably has an HLB of between about 8 and about 12.

Examples of such hydrophilic nonionic surfactants that may be mentioned include the following compounds: sorbitan monostearate polyoxyethylenated with 4 EO, sorbitan tristearate polyoxyethylenated with 20 EO the monostearate polyoxyethylenated with 8 EO hexaglyceryl monostearate, the monostearate polyoxyethylenated with 10 EO, the distearate polyoxyethylenated with 12 EO, and methylglucose distearate polyoxyethylenated with 20 EO.

The coating according to this fourth aspect of the invention of the oily globules preferably requires the use of a total amount of hydrophilic nonionic surfactant, of lipophilic nonionic surfactant and of ionic surfactant in accordance with the invention of between about 2% and about 6% by weight relative to the total weight of the composition. Even more preferentially, this amount is between 3% and 4%. The relative amounts of lipophilic, hydrophilic and ionic surfactant in accordance with the invention preferably vary within the following respective ranges: 35-55%/25-40%/15-35% by weight relative to their total weight.

The fatty phase, i.e. the coated oily droplets, preferably represents 5% to 50% by weight relative to the total weight of the composition. Even more preferentially, this percentage is between 10% and 40%. Preferably, the oil/water weight ratio is less than or equal to 1.

The weight ratio of the oily globules to the constituent elements of the coating is preferably from 2 to 13; even more preferentially, this ratio is from 6 to 8 and is especially equal to about 7.

According to one embodiment, the emulsion has a pH ranging from 5.5 to 7.5.

These compositions may also comprise at least one additional cosmetic or dermatological lipophilic active agent.

For the purposes of this fourth aspect of the present invention, the term “lipophilic active compound” means the active compound per se when it is itself an oil, or, if it is not, the active compound dissolved in an oil. The oils that may be used are the oils conventionally serving as support in cosmetic compositions, for instance short-chain fatty acid triglycerides, silicone oils, etc.

Such compositions may be obtained, for example, according to the preparation process described in patent application EP 0 705 593.

The composition according to this fourth aspect of the invention may also comprise at least one organic photoprotective agent and/or at least one mineral photoprotective agent that is active in the UVA and/or UVB range (absorbers), which may be water-soluble or liposoluble, or else insoluble in the commonly used cosmetic solvents as defined previously for the first aspect of the invention.

This fourth aspect of the invention is more particularly illustrated by Examples 13 and 14.

The examples below are given as illustrations of the invention and should not be interpreted as limiting its scope.

### EXAMPLE 1

### Preparation of the trifluoroacetic acid salt of the isopropyl monoester of N,N′-bis(benzypethylenediamine-N,N′-diacetic acid

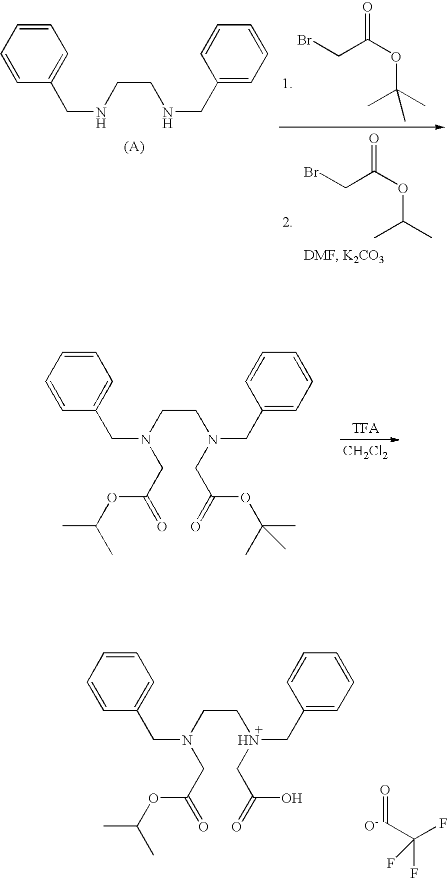

Benzathine (A) (36.19 g; 150 mmol) in 300 ml of dimethylformamide and potassium carbonate (20.81 g; 150 mmol) were placed in a 2-litre reactor equipped with a mechanical stirrer. The reaction medium was maintained at 0° C., and tert-butyl bromoacetate (27.26 g; 150 mmol) prediluted in 500 ml of dimethylformamide was then added dropwise over 5 hours. The mixture was stirred for 30 minutes, then isopropyl bromoacetate (29.37 g; 150 mmol) was added in a single portion. The reaction medium was warmed to room temperature and stirred for 17 hours. After filtration, the filtrate obtained from the reaction was concentrated. The product obtained was taken up in 150 ml of dichloromethane. The salts formed were filtered off and the filtrate was concentrated under reduced pressure.

This intermediate was dissolved in 50 ml of dichloromethane, and a 75% solution of trifluoroacetic acid in dichloromethane was added. After 6 hours, when all of the mixed diester had disappeared (bulk monitoring), the reaction medium was concentrated under reduced pressure and co-evaporated twice with toluene.

This crude product was purified on a sinter with silica, using a dichloro-methane/methanol eluent system. The percentage of methanol varied from 0% to 8%. In the first stage, the cleanest fractions were pooled and dried. A tacky brown oil was obtained, which was taken up in heptane overnight, and the solid obtained was then filtered off. This solid was recrystallized from a minimum amount of water: a first fraction (crop 1) of 3.1 g of a white powder was obtained.

In a second stage, the other fractions were pooled. An orange-yellow oil was obtained, which was recrystallized from isopropyl ether and a few millilitres of heptane. The precipitate was filtered off to obtain a white powder, which was washed with water. A second fraction (crop 2) of 3 g of a white powder was obtained. The overall yield is 10%. The product is obtained in the form of the trifluoroacetic acid salt (mono-salt).

Elemental analyses:

Calculated: C, 58.6%; H, 6.1%; N, 5.5%; O: 18.7%; F: 11.1%

Measured (crop 1): C, 58.32%; H, 6.10%; N, 5.39%; F: 10.94%

Measured (crop 2): C, 58.42%; H: 6.12%; N, 5.45%; F: 11.09%

### EXAMPLE 2

### Solubility Test

The solubility tests were performed with the diisopropyl ester of N,N′-bis(benzyl)ethylenediamine-N,N′-diacetic acid.

The test solvent was poured into a beaker, an amount of the ester compound was added with magnetic stirring and the mixture was left to stand for between 1 hour and 24 hours. The mixture was then heated to 50° C. and then cooled to room temperature (25° C.) over 24 hours. The system was then observed to see if the amount of the ester compound introduced recrystallized or remained dissolved.

The amount of ester compound that can be dissolved in the test solvent was determined in this manner. The maximum value corresponds to the amount at and above which the ester compound stops dissolving in the evaluated solvent.

The following results in several evaluated solvents were obtained:

| | Weight % of dissolved |
|---|---|
| Solvent | ester compound |
| Water | Insoluble |
| Glycerol | 1 |
| Propylene glycol | 1 |
| Butylene glycol | 1 |
| Cyclopentasiloxane | 4.5 |
| Cyclohexasiloxane | 4.7 |
| Ethanol | 8 |
| Hydrogenated isoparaffin (Parleam from NOF) | 9 |
| Squalane | 9 |
| Isopropyl palmitate | 9 |
| 2-Octyldodecanol | 10 |
| 2-Ethylhexyl salicylate | 10 |
| Dipropylene glycol | 10.7 |
| Isopropyl myristate | 11.5 |
| Hexylene glycol | 11.5 |
| Phenyl trimethicone (Dow Corning 556) | 12 |
| Apricot oil | 12 |
| Isononyl isononanoate | 16 |
| Isopropyl N-lauroylsarcosinate | 18 |
| (Eldew SL-205 ® from Ajinomoto) | |
| Dimethyl isosorbide | 38 |

It was thus found that this compound was better dissolved in isononyl isononanoate, isopropyl N-lauroylsarcosinate and dimethyl isosorbide.

### EXAMPLE 3

### Facial Care Cream

### Composition α:

| | | |
|---|---|---|
| Phase A: | | |
| Glycerol | 5 | g |
| Preserving agents (mixture of parabens - Phenonip from | 1 | g |
| Clariant) | | |
| Water | qs 100 | g |
| Phase B1: | | |
| Sorbitan tristearate (Span 65 V from Croda) | 0.9 | g |
| Glyceryl mono/distearate (36/64)/potassium stearate | 3 | g |
| mixture (Tegin Pellets from Goldschmidt) | | |
| Polyethylene glycol stearate (40 EO) | 2 | g |
| Cetyl alcohol | 4 | g |
| Stearyl alcohol | 1.20 | g |
| Phase B2: | | |
| Dibutylpentaerythrityl tetrahydroxycinnamate | 0.20 | g |
| (Tinogard TT from Ciba) | | |
| Hydrogenated polyisobutene (Parleam from NOF) | 5 | g |
| Cyclopentasiloxane | 10 | g |
| Isopropyl N-lauroylsarcosinate | 5 | g |
| Diisopropyl ester of N,N′-bis(benzyl)ethylenediamine- | 1 | g |
| N,N′-diacetic acid | | |

Phase B1 was heated to 75° C. and phase B 2 was heated to 50° C. Phase B2 was poured into phase B1. Phase A was heated to 65° C. The phase (B1+B2) was poured into phase A with stirring and emulsified at 65° C. The emulsion obtained is a waxy cream which, when applied to the face, can protect the skin against oxidative stress caused by ultraviolet rays.

A comparative composition (composition β), similar to composition a but not containing any isopropyl N-lauroylsarcosinate (replaced with 5 g of water), was also prepared according to this same protocol.

After storing compositions α and β for 2 months at 4° C., at room temperature and at 45° C., they were observed by microscope using an optical microscope of the Leica DMLB type equipped with a Sony CCD-TRIS camera, at a magnification of ×10 and in polarized light.

It was found that composition a according to the invention is homogeneous, whereas composition β contains crystals due to poor dissolution of the diisopropyl ester of N,N′-bis(benzyl)ethylenediamine-N,N′-diacetic acid.

Thus, the presence of isopropyl N-lauroylsarcosinate allows good dissolution, maintained over time, of the diisopropyl ester of N,N′-bis(benzyl)ethylenediamine-N,N′-diacetic acid.

### EXAMPLE 4

### Facial Care Cream

| | | |
|---|---|---|
| Phase A: | | |
| Propylene glycol | 3 | g |
| Preserving agents (mixture of parabens - Nipastat from | 0.3 | g |
| Clariant) | | |
| 2-Phenoxyethanol | 0.7 | g |
| Water | 63.7 | g |
| Phase B1: | | |
| Sorbitan tristearate (Span 65 V from Croda) | 0.9 | g |
| Glyceryl mono/distearate (36/64)/potassium stearate | 3 | g |
| mixture (Tegin Pellets from Goldschmidt) | | |
| Polyethylene glycol stearate (40 EO) | 2 | g |
| Cetyl alcohol | 4 | g |
| Stearic acid | 1.20 | g |
| Hydrogenated polyisobutene (Parleam from NOF) | 5 | g |
| Phase B2: | | |
| Cyclopentasiloxane | 10 | g |
| Phase B3: | | |
| Dibutylpentaerythrityl tetrahydroxycinnamate | 0.20 | g |
| (Tinogard TT from Ciba) | | |
| Dimethyl isosorbide | 5 | g |
| Diisopropyl ester of N,N′-bis(benzyl)ethylenediamine- | 1 | g |
| N,N′-diacetic acid | | |

Phase B1 was heated to 75° C. and phase B2 was heated to 50° C. along with phase B3. Phase B2 was poured into phase B1, and B3 was then added. Phase A was heated to 65° C. The phase (B1+B2) was poured into phase A with stirring and emulsified at 65° C. The emulsion obtained is a waxy cream which, when applied to the face, can protect the skin against oxidative stress caused by ultraviolet rays.

After storage for 2 months (4° C., room temperature and 45° C.), no recrystallization of the diisopropyl ester of N,N′-bis(benzyl)ethylenediamine-N—N′ diacetic acid in this composition was observed.

### EXAMPLE 5

### Facial Care Cream

| | | |
|---|---|---|
| Phase A: | | |
| Crosslinked acrylic acid polymer (Carbopol 931 from | 0.3 | g |
| Noveon) | | |
| Triethanolamine | 0.3 | g |
| Preserving agents (mixture of parabens - Nipastat from | 0.3 | g |
| Clariant) | | |
| Water | 67.9 | g |
| Phase B1: | | |
| Mixture of glyceryl stearate and PEG-100 stearate | 2.5 | g |
| (Arlacel ® 165 FL from Uniqema) | | |
| Polyethylene glycol stearate (50 EO) | 2.5 | g |
| Stearyl alcohol | 1 | g |
| Cetyl alcohol | 1 | g |
| Hydrogenated polyisobutene (Parleam from NOF) | 5 | g |
| Phase B2: | | |
| Cyclopentasiloxane | 12 | g |
| Phase B3: | | |
| Dibutylpentaerythrityl tetrahydroxycinnamate | 0.20 | g |
| (Tinogard TT from Ciba) | | |
| Isononyl isononanoate | 6 | g |
| Diisopropyl ester of N,N′-bis(benzyl)ethylenediamine- | 1 | g |
| N,N′-diacetic acid | | |

The composition was prepared in the same manner as for the composition of Example 2.

After 2 months of storage (4° C., room temperature and 45° C.), no recrystallization of the diisopropyl ester of N,N′-bis(benzyl)ethylenediamine-N—N′-diacetic acid in this composition was observed.

When applied to the face, the composition protects the skin against the oxidative stress caused by ultraviolet rays.

### EXAMPLE 6

An antisun composition containing the following ingredients is prepared:

| | | |
|---|---|---|
| Propylene glycol | 6 | g |
| Glycerol | 6 | g |
| Pentasodium salt of ethylenediaminetetraacetic acid | 0.1 | g |
| Water | qs 100 | g |
| 2-Ethylhexyl 2-cyano-3,3-diphenylacrylate | 10 | g |
| 4-tert-Butyl-4′-methoxydibenzoylmethane at 33% in water | 2.5 | g |
| Drometrizole trisiloxane (Silatrizole from Rhodia) | 4 | g |
| Ethylhexyl triazone (Uvinul T150 from BASF) | 2 | g |
| Isopropyl N-lauroylsarcosinate | 5 | g |
| Dibutylpentaerythrityl tetrahydroxycinnamate | 0.20 | g |
| (Tinogard TT from Ciba) | | |
| Diisopropyl ester of N,N′-bis(benzyl)ethylenediamine- | 1 | g |
| N,N′-diacetic acid | | |
| Mixture of tocopherols in soybean oil (50/50) | 0.2 | g |
| (COVI - OX T 50 C from Cognis) | | |
| Mixture of dimethicone copolyol, cyclopentasiloxane and | 1 | g |
| water (10/88/2) (DC 5225C from Dow Corning) | | |
| α,ω-Dihydroxylated polydimethylsiloxane/cyclo- | 2 | g |
| pentadimethyl-siloxane mixture (14.7/85.3) | | |
| (Dow Corning 1501 FL from Dow Corning) | | |
| Glyceryl 2-ethylhexyl ether | 0.5 | g |
| Titanium dioxide (15 nm) treated with aluminium stearate/ | 1 | g |
| alumina (MT-100 T V from Tayca) | | |
| Acrylic acid/stearyl methacrylate copolymer polymerized | 0.4 | g |
| in an ethyl acetate/cyclohexane mixture (Pemulen TR-1 | | |
| Polymer from Noveon) | | |
| Cyclohexasiloxane | 3 | g |
| Triethanolamine | 0.4 | g |
| Ethanol | 6 | g |

### EXAMPLE 7

### Comparative

The following oil-in-water emulsions were prepared:

| | α | |
|---|---|---|
| | (control) | β |
| Glyceryl stearate | 3 | 3 |
| Polyethylene glycol stearate (40 OE) | 2 | 2 |
| Sorbitan tristearate (Span 65 V from Croda) | 0.90 | 0.90 |
| Cetyl alcohol | 4 | 4 |
| Sodium stearoylglutamate | 0 | 1.2 |
| (Amisoft HS 11 PF from Ajinomoto) | | |
| Stearic acid | 1.2 | 0 |
| Hydrogenated polyisobutene (Parleam from NOF) | 5 | 5 |
| Isopropyl N-lauroylsarcosinate | 5 | 5 |
| Diisopropyl ester of N,N′- | 1 | 1 |
| bis(benzyl)ethylenediamine-N,N′-diacetic acid | | |
| Dibutylpentaerythrityl tetrahydroxycinnamate | 0.2 | 0.2 |
| (Tinogard TT from Ciba) | | |
| Cyclopentasiloxane | 10 | 10 |
| Propylene glycol | 3 | 3 |
| Mixture of methyl, butyl, ethyl, propyl and isobutyl | 0.3 | 0.3 |
| p-hydroxybenzoates (7/57/22/14) | | |
| Triethanolamine | 0.3 | 0 |
| Water | qs 100 | qs 100 |

The content of the diisopropyl ester of N,N′-bis(benzyl)ethylenediamine-N,N′-diacetic acid after storage for 2 months was then measured, at 4° C., at room temperature and at 45° C.

The content of diester compound is measured by reverse-phase high performance liquid chromatography using a Waters/X-Trerra RP18 (5 μm) −150 nm column, using, as eluent, a 60/40 and 95/5 gradient of acetonitrile/0.02 M sodium acetate buffer.

The chromatographic conditions are as follows:

flow rate: 1 ml/ml

injected volume: 20 μl

detection: 210 nm

retention time: about 6.7 minutes

The two calibration solutions used are the following: 12 μg/ml and 120 μg/ml of the diisopropyl ester of N,N′-bis(benzyl)ethylenediamine-N,N′-diacetic acid in acetonitrile.

Preparation of the Sample to be Measured (Test Solution):

A mass m of stored composition is dispersed in a volume V of acetonitrile so as to obtain a concentration of diester compound that is within the calibration range.

The calibration curve is plotted, placing the surface area values obtained on the chromatograms of the calibration solutions on the y-axis, and the concentrations of diester compound in the calibration solutions on the x axis.

The concentration in the stored composition is deduced from the calibration curve by using the following formula: C=(S−A)/B,
C being the concentration of diester compound in the test solution (in mg/ml)
S being the surface area of the peak for the diester compound in the test chromatogram
A being the y-axis value at the origin of the calibration curve for the diester compound, and
B being the slope of the calibration curve for the diester compound.

The content T (in %) of diester compound in the stored solution is determined by the following formula: T=100×(C×V)/m,
C being the concentration of diester compound in the test solution (in mg/ml),
V being the final volume after dispersion of the sample (in ml), and
m being the amount of sample of the stored composition.

The results are as follows:

| Content after 2 months | α | |
|---|---|---|
| of storage | (control) | β |
| at 4° C. | 1.0% | 1.0% |
| at 45° C. | 0.8% | 0.9% |

The use of an ionic surfactant in accordance with this second aspect of the invention of sodium stearoylglutamate type makes it possible to maintain, at elevated temperature, the chemical stability of the diisopropyl ester of N,N′-bis(benzyl)ethylenediamine-N,N′-diacetic acid in an oil-in-water emulsion.

### EXAMPLE 8

An antisun composition in the form of an oil-in-water emulsion containing the following ingredients is prepared:

| | | |
|---|---|---|
| Triethanolamine | 0.88 | g |
| Disodium salt of ethylenediaminetetraacetic acid | 0.1 | g |
| Dibutylpentaerythrityl tetrahydroxycinnamate | 0.20 | g |
| (Tinogard TT from Ciba) | | |
| Isopropyl N-lauroylsarcosinate | 8 | g |
| Diisopropyl ester of N,N′-bis(benzyl)ethylene- | 1 | g |
| diamine-N,N′-diacetic acid | | |
| Stearyl alcohol | 1 | g |
| Preserving agents (mixture of parabens and | 1 | g |
| phenoxyethanol - Phenonip from Clariant) | | |
| Preserving agents (mixture of parabens -Nipastat | 0.25 | g |
| from Clariant) | | |
| 2-Ethylhexyl salicylate | 5 | g |
| 4-tert-Butyl-4′-methoxydibenzoylmethane at | 3 | g |
| 33% in water | | |
| Terephthalylidenedicamphorsulfonic acid | 5 | g |
| 2-Ethylhexyl 2-cyano-3,3-diphenylacrylate | 7 | g |
| Drometrizole trisiloxane (Silatrizole from Rhodia) | 1.5 | g |
| Acrylamide/sodium acrylamido-2-methylpropanesulfonate | 2 | g |
| as a 40% inverse emulsion in isohexadecane/water | | |
| (Simulgel 600 from SEPPIC) | | |
| Styrene/acrylate copolymer particles (Sunspheres | 2 | g |
| Powder from Rohm & Haas) | | |
| Cyclopentasiloxane | 3 | g |
| Glycerol | 5 | g |
| Mixture of glyceryl stearate and PEG-100 stearate | 2 | g |
| (Arlacel ® 165 FL from Uniqema) | | |
| Sodium stearoylglutamate (Amisoft HS 11 PF from | 1.3 | g |
| Ajinomoto) | | |
| Water | qs 100 | g |

### EXAMPLE 9

### Oil-in-water (O/W) Emulsion Stabilized with an Amphiphilic AMPS Polymer in Accordance with the Third Aspect of the Invention

| Phase A: | |
|---|---|
| Glycerol | 5% |
| (AMPS/ethoxylated C₁₂/C₁₄ alcohol methacrylate) | 1% |
| copolymer (8 mol EO) (80/20) (Aristoflex LNC ® | |
| from Clariant) | |
| Triethanolamine | 0.0060% |
| Preserving agents | 1% |
| Diisopropyl ester of N,N′-bis(benzyl)ethylenediamine- | 1% |
| N-N′-diacetic acid | |
| Water | qs 100.00 |
| Phase B: | |
| Dibutylpentaerythrityl tetrahydroxycinnamate | 0.20% |
| (Tinogard TT from Ciba) | |
| Hydrogenated isoparaffin (Parleam from NOF | 7% |
| Corporation) | |
| Cyclopentasiloxane | 8% |
| Glyceryl monoisostearate | 0.50% |

Phase A is brought to 60° C. and stirred using a deflocculator at 450 rpm for 1 hour. Phase B is brought to 60° C. in order for the solid constituents to be dissolved. Phase B is then poured into phase A at about 60° C.

The emulsion obtained is a moisturizing milk, whose application to the face protects the skin against the oxidative stress caused by ultraviolet rays.

After storing this composition for 2 months at 4° C., at room temperature and at 45° C., an observation by microscope was performed using an optical microscope of the Leica DMLB type equipped with a Sony CCD-IRIS camera, at a magnification of ×10 and in polarized light.

No recrystallization of the diisopropyl ester of N,N′-bis(benzyl)ethylene-diamine-N—N′-diacetic acid in this composition was observed.

Thus, the presence of an effective amount of an AMPS polymer in accordance with the third aspect of the invention allows good introduction, maintained over time, of the diisopropyl ester of N,N′-bis(benzyl)ethylenediamine-N—N′-diacetic acid into the oily phase of the emulsion.

### EXAMPLE 10

### Comparative

### Oil-in-water (O/W) Emulsion Stabilized with an Emulsifying Polymer of the acrylates/C₁₀-C₃₀ alkyl acrylate Crosslinked Polymer Type

| Phase A: | |
|---|---|
| Glycerol | 5% |
| Acrylates/C₁₀-C₃₀ alkyl acrylate crosslinked polymer | 1% |
| (Carbopol ® Ultrez 20 Polymer from Noveon) | |
| Triethanolamine | 0.98% |
| Preserving agents | 1% |
| Diisopropyl ester of N,N′-bis(benzyl)ethylenediamine- | 1% |
| N-N′-diacetic acid | |
| Water | qs 100.00 |
| Phase B: | |
| Dibutylpentaerythrityl tetrahydroxycinnamate | 0.20% |
| (Tinogard TT from Ciba) | |
| Hydrogenated isoparaffin (Parleam from NOF | 7% |
| Corporation) | |
| Cyclopentasiloxane | 8% |
| Glyceryl monoisostearate | 0.50% |

This composition was obtained by following the same experimental protocol as that indicated for Example 9.

After only 24 hours, recrystallization of the diisopropyl ester of N,N′-bis(benzyl)ethylenediamine-N—N′-diacetic acid is observed.

### EXAMPLE 11

### Oil-in-water (O/W) Emulsion Stabilized with an Emulsifying Polymer of Hydrophobic Modified Cellulose Type.

| Phase A: | |
|---|---|
| Glycerol | 5% |
| Cetylhydroxyethylcellulose (Natrosol ® Plus CS 330 from | 1% |
| Hercules) | |
| Preserving agents | 1% |
| Diisopropyl ester of N,N′-bis(benzyl)ethylenediamine- | 1% |
| N-N′-diacetic acid | |
| Water | qs 100.00 |
| Phase B: | |
| Dibutylpentaerythrityl tetrahydroxycinnamate | 0.20% |
| (Tinogard TT from Ciba) | |
| Hydrogenated isoparaffin (Parleam from NOF | 7% |
| Corporation) | |
| Cyclopentasiloxane | 8% |
| Glyceryl monoisostearate | 0.50% |

This composition was obtained by following the same experimental protocol as that indicated for Example 9.

After only 24 hours, recrystallization of the diisopropyl ester of N,N′-bis(benzyl)ethylenediamine-N—N′-diacetic acid is observed.

### EXAMPLE 12

### Oil-in-water (O/W) Emulsion Stabilized with an Amphiphilic AMPS Polymer in Accordance with the Third Aspect of the Invention

| Phase A: | |
|---|---|
| Glycerol | 5% |
| (AMPS/ethoxylated C12/C14 alcohol methacrylate) | 1% |
| copolymer (8 mol EO) (80/20) (Aristoflex LNC ® | |
| from Clariant) | |
| Triethanolamine | 0.006% |
| Preserving agents | 1% |
| Diisopropyl ester of N,N′-bis(benzyl)ethylenediamine- | 1% |
| N-N′-diacetic acid | |
| Water | qs 100.00 |
| Phase B: | |
| Dibutylpentaerythrityl tetrahydroxycinnamate | 0.20% |
| (Tinogard TT from Ciba) | |
| 2-Ethylhexyl cyano-3,3-diphenylacrylate | 3% |
| Hydrogenated polyisobutene (Parleam from NOF | 7% |
| Corporation) | |
| Cyclopentasiloxane | 8% |
| Glyceryl monoisostearate | 0.50% |

This composition was obtained by following the same experimental protocol as that indicated for Example 9.

No recrystallization of the diisopropyl ester of N—N′bis(benzyl)ethylene-diamine-N—N′-diacetic acid in this composition is observed.

When applied to the face, the composition can protect the skin against UV rays.

### EXAMPLE 13

### Oil-in-water (O/W) Emulsion in the Form of Oleosomes

A facial care cream having the following composition α was prepared:

| | | |
|---|---|---|
| Phase A: | | |
| Polyoxyethylenated sorbitan monostearate (4 EO) | 1.76 | g |
| (Tween 61 V from Croda) | | |
| Sucrose tristearate (Ryoto Sugar Ester S 370 from | 2.64 | g |
| Mitsubishi Kagaku Foods) | | |
| Stearyl alcohol | 1.21 | g |
| Cholesterol | 0.05 | g |
| Isostearyl stearate | 5 | g |
| Hydrogenated polyisobutene (Parleam from NOF) | 7 | g |
| Cyclopentasiloxane | 3 | g |
| Sodium stearylglutamate (Amisoft HS 11 PF from | 0.25 | g |
| Ajinomoto) | | |
| Diisopropyl ester of N,N′-bis(benzyl)ethylenediamine- | 1 | g |
| N,N′-diacetic acid | | |
| Dibutylpentaerythrityl tetrahydroxycinnamate | 0.20 | g |
| (Tinogard TT from Ciba) | | |
| Tocopherol | 0.20 | g |
| Phase B: | | |
| Disodium salt of ethylenediaminetetraacetic acid | 0.1 | g |
| 2-Phenoxyethanol | 0.50 | g |
| Butyl paraben | 0.10 | g |
| Methyl paraben | 0.25 | g |
| Chlorphenesin | 0.25 | g |
| Water | qs 100 | g |
| Phase C: | | |
| Water | 13 | g |
| Carboxyvinyl polymer (Synthalen K from 3V) | 0.40 | g |
| Phase D: | | |

| | |
|---|---|
| Triethanolamine | 0.40% |

Phase A was heated until all the fatty substances had fully melted (about 85° C.).

Phase B was heated to between 80° C. and 85° C. Phase B was added to phase A with very vigorous stirring. The emulsion was then subjected to high-pressure homogenization. Phases C and D were finally added.

The emulsion obtained in the form of oleosomes has a pH equal to 6.10.

A similar composition β was prepared by replacing the sodium stearoyl-glutamate with potassium cetyl phosphate (Amphisol K® from DSM Nutritional Products) (0.25 g) and another composition γ (control) was prepared by replacing the sodium stearoylglutamate with stearic acid (1.32 g; with corresponding reduction of the amount of water).

The content of diisopropyl ester of N,N′-bis(benzyl)ethylenediamine-N,N′-diacetic acid after 2 months of storage at 4° C. and at 45° C. was then measured.

The content of diester compound is measured by reverse-phase high performance liquid chromatography using a Waters/X-Terra RP18 (5 μm)-150 nm column, using, as eluent, a 60/40 and 95/5 gradient of acetonitrile/0.02 M sodium acetate buffer.

The chromatographic conditions are as follows:

flow rate: 1 ml/ml

injected volume: 20 μl

detection: 210 nm

retention time: about 6.7 minutes

The two calibration solutions used are as follows: 12 μg/ml and 120 μg/ml of the diisopropyl ester of N,N′-bis(benzyl)ethylenediamine-N,N′-diacetic acid in acetonitrile.

Preparation of the Sample to be Measured (Test Solution):

A mass m of the stored composition is dispersed in a volume V of acetonitrile so as to obtain a concentration of diester compound that is within the calibration range.

The calibration curve is plotted, placing the surface area values obtained on the chromatograms of the calibration solutions on the y-axis, and the concentrations of diester compound in the calibration solutions on the x axis.

The concentration in the stored composition is deduced from the calibration curve by using the following formula: C=(S−A)/B,
C being the concentration of diester compound in the test solution (in mg/ml)
S being the surface area of the peak for the diester compound in the test chromatogram
A being the y-axis value at the origin of the calibration curve for the diester compound, and
B being the slope of the calibration curve for the diester compound.

The content T (in %) of diester compound in the stored solution is determined by the following formula: T=100×(C×V)/m,
C being the concentration of diester compound in the test solution (in mg/ml),
V being the final volume after dispersion of the sample (in ml), and
m being the amount of sample of the stored composition.

The results are as follows:

| Content after 2 months | | | γ |
|---|---|---|---|
| of storage | α | β | (control) |
| at 4° C. | 1.0% | 1.0% | 1.0% |
| at 45° C. | 1.0% | 1.0% | 0.8% |

It is found that the use especially of an ionic surfactant in accordance with the fourth aspect of the invention of sodium stearoylglutamate or potassium cetyl phosphate type makes it possible to maintain, at elevated temperature, the chemical stability of the diisopropyl ester of N,N′-bis(benzyl)ethylenediamine-N,N′-diacetic in an oil-in-water emulsion that is in the form of oleosomes.

### EXAMPLE 14

### Oil-in-Water (O/W) Emulsion in the Form of Oleosomes

A facial care cream having the following composition is prepared:

| | | |
|---|---|---|
| Phase A: | | |
| Water | 39.25 | g |
| Methyl paraben | 0.25 | g |
| Glycerol | 5 | g |
| 2-Phenoxyethanol | 0.7 | g |
| Disodium salt of ethylenediaminetetraacetic acid | 0.2 | g |
| 1,2-Octanediol | 0.4 | g |
| Phase B: | | |
| Polyoxyethylenated sorbitan monostearate (4 EO) | 1 | g |
| (Tween 61 V from Croda) | | |
| Sucrose tristearate (Ryoto Sugar Ester S 370 from | 2 | g |
| Mitsubishi Kagaku Foods) | | |
| Sodium stearoylglutamate (Amisoft HS 11 PF from | 0.75 | g |
| Ajinomoto) | | |
| Butyl paraben | 0.15 | g |
| Cyclohexasiloxane | 1.7 | g |
| Diisopropyl sebacate | 2.7 | g |
| Isononyl isononanoate | 5.6 | g |
| Diisopropyl ester of N,N′-bis(benzyl)ethylenediamine- | 1 | g |
| N,N′-diacetic acid | | |
| 2-Ethylhexyl cyano-3,3-diphenylacrylate | 7 | g |
| 4-tert-Butyl-4′-methoxydibenzoylmethane | 3 | g |
| 2-Ethylhexyl salicylate | 5 | g |
| Phase C: | | |
| Water | qs 100 | g |
| Carboxyvinyl polymer (Synthalen K from 3V) | 0.30 | g |
| Triethanolamine | 0.3 | g |
| Phase D: | | |
| Water | 6 | g |
| Xanthan gum | 0.2 | g |
| Phase E: | | |
| Corn starch esterified with octenylsuccinic | 3 | g |
| anhydride, aluminium salt (Dry Flo Plus from | | |
| National Starch) | | |
| Phase F: | | |
| PEG-12 dimethicone (Silsoft ® 880 from | 0.5 | g |
| General Electric) | | |

Phase A is heated to about 60-65° C. and phase B to about 80° C. Phase A is poured into phase B very quickly while blending (phase inversion) and while maintaining at 60-65° C., and the mixture is emulsified for 15 minutes. At room temperature, phases C, D, E and F are successively added.

1. Cosmetic and/or dermatological composition comprising, in a physiologically acceptable medium containing at least one oil:
(a) at least one compound of general formula (Ia): 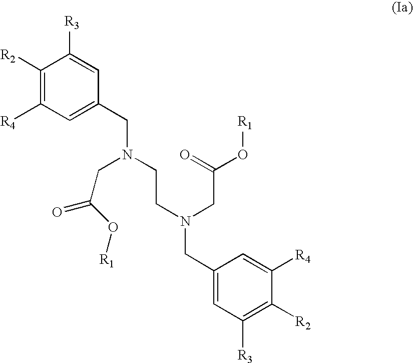
in which:
R₁ represents a saturated linear alkyl radical containing from 1 to 6 carbon atoms or a saturated branched alkyl radical containing from 3 to 6 carbon atoms,
R₂, R₃ and R₄ represent, independently of each other, a hydrogen atom or a radical —OR₅, and
R₅ represents a hydrogen atom or a saturated linear alkyl radical containing from 1 to 5 carbon atoms or a saturated branched alkyl radical containing from 3 to 5 carbon atoms,
or salts thereof, and
(b) an effective amount of at least one solvent chosen from:
(i) isononyl isononanoate;
(ii) dimethyl isosorbide;
(iii) amino acid esters of formula (II):
R′₁(CO)N(R′₂)CH(R′₃)(CH₂)ₙ(CO)OR′₄ (II)
in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical, and
R′₄ represents a linear or branched C₁ to C₁₀ alkyl radical, a linear or branched C₂ to C₁₀ alkenyl radical or a sterol residue; and
(iv) a mixture thereof.

2. Composition according to claim 1, in which R₂, R₃ and R₄ represent a hydrogen atom.

3. Composition according to claim 1, in which R₁ denotes an isopropyl radical.

4. Composition according to claim 1, in which the compound of general formula (Ia) is the diisopropyl ester of N,N′-bis(benzyl)ethylenediamine-N,N′-diacetic acid.

5. Composition according to claim 1, comprising from 0.01% to 5% by weight of compound of general formula (Ia) relative to the total weight of the said composition.

6. Composition according to claim 1, in which the said amino acid ester is isopropyl N-lauroylsarcosinate of formula CH₃—(CH₂)₁₀CO—N(CH₃)—CH₂—COO—CH(CH₃)₂.

7. Composition according to claim 1, comprising from 0.05% to 25% by weight of the said solvent relative to the total weight of the composition.

8. Composition according to claim 1, in which the said solvent and the said compound of general formula (Ia) are present in a [solvent/compound of general formula (Ia)] mass ratio ranging from 1.5 to 15.

9. Composition according to claim 1, in which the said solvent is isononyl isononanoate and the [isononyl isononanoate/compound of general formula (Ia)] mass ratio is ranging from 5.25 to 8.

10. Composition according to claim 1, in which the solvent is an amino acid ester of formula (II) and the [amino acid ester of formula (1)/compound of general formula (Ia)] mass ratio is ranging from 4.5 to 10.

11. Composition according to claim 1, characterized in that it is in the form of a water-in-oil or oil-in-water emulsion.

12. Composition according to claim 1, also comprising at least one organic photoprotective agent and/or at least one mineral photoprotective agent that is active in the UVA and/or UVB range.

13. Non-therapeutic treatment process for caring for and/or making up keratin materials, comprising at least the step of applying to the said keratin materials, at least one composition as defined in claim 1.
